(19) United States
(12) Patent Application Publication
(10) Pub. No.: US 2023/0329955 A1
(43) Pub. Date: Oct. 19, 2023

(54) DEEP TISSUE AND/OR MYOFASCIAL TREATMENT OF A PATIENT BY IMPARTING A FORCE ON A PRESSURE HEAD TO A TISSUE OF THE PATIENT

(71) Applicant: Novasana LLC, Grants Pass, OR (US)

(72) Inventors: Nicholas Baida, Grants Pass, OR (US); Julian Cangilose, Grants Pass, OR (US); Lisa Kissel, Grants Pass, OR (US)

(21) Appl. No.: 17/722,124

(22) Filed: Apr. 15, 2022

(51) Int. Cl. A61B 6/04; A61H 1/00; A61H 7/00

(52) U.S. Cl. CPC A61H 1/008; A61B 6/0407; A61H 7/007; A61N 2007/0034

(57) ABSTRACT

Disclosed is a method, a device, a system and/or a manufacture of deep tissue and/or myofascial treatment of a patient by imparting a force on a pressure head to a tissue of the patient. In one embodiment, a device for massage and/or physical therapy includes a pressure head protruding below a lever arm for applying a pressure to a tissue of a patient. A force acting on the lever arm multiplies the force applied to the tissue by the pressure head. A first hinge is attached to a first end of the lever arm and configured to allow the pressure head to pivot into and out of contact with the tissue of the patient. The support is coupled to the first hinge elevating the first hinge above a fastening point. A fastener coupled to the support can fix the device when the pressure is applied to the tissue of the patient.

### FIELD OF TECHNOLOGY

This disclosure relates generally to medical devices and, more particularly, to a method, a device, and/or a system of deep tissue and/or myofascial treatment of a patient by imparting a force on a pressure head to a tissue of the patient.

### BACKGROUND

Injury to muscle tissue, joint tissue, and other deep tissue is common for the human body. Such can arise for a variety of reasons, and may be encountered by everyone from athletes who frequently exert their bodies to everyday workers who may hold low-level muscle tension for long periods such as while using computers at desks. Pain and injury of muscles, joints, or other deep tissue may arise from physical trauma, general inflammatory responses, and/or surgical procedures.

For example, one source of pain may be two distinct but related phenomenon of muscle and its surrounding connective tissue, the “fascia”. The fascia may be a web-like connective tissue, which may be primarily made of collagen, located beneath the skin. The fascia may attach, stabilize, enclose, and/or separate muscles and other internal organs. The fascia also may bundle individual muscle fibers or cells to import shape to muscle tissue. The fascia is generally a robust material, capable of enduring high stress and strain. However, during muscle use, knots or tangles in the connective tissue can form as the fascia adheres to itself. Over time these tangles may increase, and may cause restriction in muscles and pain. The knots can lead to tightness of the muscle, pain (including extreme or chronic pain), pain in joints and other deep tissue, and/or limited range of motion. As another example source of pain is injury to tendons, nerve compression, joint surface misalignment and/or compression, and ligament injuries.

Manual pressure, manipulation, and/or massage of tissue is a general treatment for the pain or injury of many muscles, joints, and other deep tissue. For example, manual pressure applied to sarcomere knots at a number of angles and pressures may initiate “myofascial release” that unwinds such knots. Manual pressure on injured muscles and joints may increase blood flow, increase the rate of healing, and/or assist in pain relief.

While massage with hands or handheld tools is one method of providing pressure and manual manipulation, some challenges may exist in its application. First, it can be difficult for a practitioner of manual manipulation (e.g., masseuse, chiropractor, physical therapist, physician, other professional healer, etc.) to apply enough pressure to sufficiently treat a target tissue. Similarly, it can be challenging to repeatedly apply enough pressure, and retain strength through multiple sessions without resting. Second, massage sessions can be highly inconsistent from one practitioner to another, one organization to another, and/or among different tools that attempt to impart pressure. Third, it can be difficult for a patient and/or practitioner to remember, record, or communicate how manual pressure treatments and/or massage was applied. This may be helpful to track progress and improve quality across more than one treatment. Similarly, it may be difficult to record or recall notes when the patient and/or practitioner must focus on providing the treatment. For example, the patient may need to focus on relaxing muscles to receive the treatment, and the practitioner may need to focus on applying the right amount of pressure to the right location. Each of such challenges may occur individually or together in combinations depending on the type of treatment prescribed or the needs of the patient.

It is desirable that new forms of providing manual pressure are invented to aid in the healing of muscles, joints, and other deep tissue including for the healing of injury, the reduction of pain, and the general comfort and wellness of patients.

### SUMMARY

Disclosed are a method, a device, and/or a system of deep tissue and/or myofascial treatment of a patient by imparting a force on a pressure head to a tissue of the patient.

In one embodiment, a device for massage, physical therapy, chiropractic therapy, and/or myofascial release includes a pressure head, a lever arm, a first hinge, a support, and a fastener. The pressure head applies a pressure to a tissue of a patient. The lever arm comprises a first end and a second end coupled to the pressure head. The pressure head protrudes below the lever arm and a force acting on the second end of the lever arm multiplies the force applied to the tissue by the pressure head.

The first hinge is attached to the first end of the lever arm and configured to allow the pressure head to pivot into and out of contact with the tissue of the patient. The support is coupled to the first hinge elevating the first hinge above a fastening point. A fastener coupled to the support can fix the device when the pressure is applied to the tissue of the patient.

A track may be configured to translate the device along a first axis, referred to as an x-axis, running parallel to a surface that is elongated and usable to receive the patient in a substantially flat position. The fastener may fix the device at a location in the track along the first axis.

The device may include a second hinge coupled to the support and configured to rotate the lever arm around a second axis, referred to as a y-axis, that runs perpendicular to the elongated surface for receiving the patient. The second hinge may enable rotation of the lever arm such that an oblique pressure can be applied from the pressure head to the tissue when the patient is positioned substantially flat on the surface.

The device may also include a third hinge coupled to the support and configured to rotate the lever arm around a third axis, referred to as a z-axis, that runs perpendicular to both the y-axis and the x-axis. The third hinge may enable rotation of the lever arm to apply the pressure to the tissue at two or more horizontal locations of the patient without the patient repositioning on the surface.

A first grip may be located on the lever arm at the second end of the lever arm and/or a first location between the pressure head and the second end of the lever arm. The first grip may be configured to receive an extremity of a practitioner to apply the force acting on the second end of the lever arm. The first grip may be positioned such that the force applied to the tissue by the pressure head is an integer multiple of the force acting on the second end of the lever arm.

The fastener may be coupled to a surface that is a table on which the patient rests in the substantially flat position. The table may include a head space comprising a cavity and a hole for receiving a head of the patient when the patient is positioned on their stomach on the surface. The track may extend along one edge of the table such that the pressure head may be translated to different locations along the table.

The device may include an attachment point located on the lever arm coupling the pressure head to the lever arm. The pressure head may be detachable from the lever arm such that a different pressure head and/or a replacement pressure head can be attached to the attachment point. The pressure head may include a friction surface for engagement with skin over the tissue and/or for gripping the skin during application of the pressure to provide a sheer pressure to the tissue. The friction surface may include a rubber, a silicone, and/or a polymer.

The device may include a second grip located on the lever arm between the pressure head and the first grip. The second grip may be used to receive an extremity of a practitioner to apply the force acting on the second end of the lever arm at a different force multiplier than the first grip. The device may also include a sensor measuring the pressure applied to the tissue of the patient and/or the force applied to the pressure head. The device may further include a sensor transmitting data to a computer memory for storage and/or a user interface for display to the practitioner.

A length of the lever arm may be adjustable to adjust a force multiplier and/or to permit a practitioner to remain in physical contact with the patient during treatment. The lever arm may include a second track allowing the pressure head to slide along the lever arm to adjust the force multiplier on the lever arm and/or reposition the pressure head enabling application of pressure to the tissue at different horizontal locations of the patient without the patient repositioning on the surface.

In another embodiment, a method for bodily tissue manipulation includes positioning a patient on a surface and placing a pressure head on a tissue of the patient. The pressure head is coupled to a lever arm pivoting on a fulcrum that is fixable relative to the surface. The lever arm includes an effort portion of the lever arm, a load portion of the lever arm, and the fulcrum comprises a pivot point. A force is applied to the lever arm at the effort portion of the lever arm; A multiplier force is applied to the pressure head at the load portion of the lever arm to provide a pressure treatment for healing the patient that is able to be duplicated while reducing exertion on a practitioner applying the force to the lever arm.

The method may also translate the lever arm and the pressure head parallel to a vertical axis of the patient and immovably fix the pressure head relative to the surface. The pivot point of the fulcrum may be rotated around an axis perpendicular to a frontal axis of the patient. The force may result in an oblique pressure applied by the pressure head. The pivot point of the fulcrum may be rotated around an axis perpendicular to a sagittal axis of the patient to reposition a contact point of the pressure head to the tissue of the patient.

The method may also change a force multiplier by adjusting a length of the effort portion of the lever arm, a position of the pressure head along the lever arm, and/or a position of the grip along the lever arm. In addition, the pressure head may be replaced with a different pressure head, and a different pressure treatment may then be applied to the patient.

The method may also record a parameter of the pressure treatment dependent on: the length of the effort portion of the lever arm, the position of the pressure head along the lever arm, the position of the grip along the lever arm, the force applied to the lever arm, the force applied by the pressure head, a location along the axis parallel to the vertical axis of the patient, a rotation around the axis perpendicular to the frontal axis of the patient, a rotation around the axis perpendicular to the sagittal axis of the patient, a time over which a pressure is applied, a rotational sheer pressure, a vibration setting, a temperature of the pressure head, and/or a type of pressure head.

The method may measure the multiplier force and/or a pressure applied by the pressure head with an electronic sensor. The method may also determine an x-translation value of the location along the axis parallel to the vertical axis of the patient, a y-rotation value of the rotation around the axis perpendicular to the frontal axis of the patient, a z-rotation value of the rotation around the axis perpendicular to the sagittal axis of the patient, and/or a type of pressure head. A computer memory may then store the multiplier force, the pressure applied by the pressure head, and the x-translation value, the y-rotation value, and/or the z-rotation value.

In yet another embodiment, a device may include a table for receiving a patient and a lever arm fixable relative to the table for multiplying a force exerted on the lever arm at a pressure head. The lever arm can be translated along at least one axis and rotated around at least one axis.

A contact point of the pressure head can be positioned at least two contact points along a surface of the table through a translation of the lever arm along an x-axis, a rotation around a y-axis, and/or a rotation around a z-axis. The x-axis may be parallel to a vertical axis of the patient when the patient lies on their stomach on the table, the y-axis may be perpendicular to the x-axis and parallel to a frontal axis of the patient when the patient lies on their stomach on the table, and/or the z-axis may be perpendicular to both the x-axis and the y-axis and parallel to a sagittal axis when the patient lies on their stomach on the table.

A length of the lever arm may be adjustable to adjust a force multiplier and/or to permit a practitioner to remain in physical contact with the patient during treatment. The lever arm may include a second track allowing the pressure head to slide along the lever arm to adjust the force multiplier on the lever arm. The pressure head may be detachable from the lever arm such that a different pressure head and/or a replacement pressure head may be attachable to an attachment point along the lever arm. The device may further include a sensor measuring the pressure applied to the tissue of the patient and/or the force applied to the pressure head. The sensor may transmit data to a computer memory for storage and/or a user interface for display to the practitioner.

In still yet another embodiment, a device for applying a pressure treatment to a tissue of a patient includes a pressure head for applying a pressure to the tissue of the patient and a head support arm coupled to the pressure head. The pressure head protrudes below the head support arm and a force acting on the head support arm applies the force to the tissue through the pressure head. A support coupled to the head support arm distances the head support arm from the patient. The device also includes a z-axis adjustor coupled to the support and the head support arm, the z-axis adjustor configured to move the head support arm such that a distance of the pressure head to the tissue of the patient may be adjusted. A force generator is coupled to the support and/or the head support arm and is configured to apply the force to the head support arm. The device also includes a fastener coupled to the support, for fixing the device when the pressure is applied to the tissue of the patient.

The force generator may include a cam that when rotating applies the force to the head support arm. The force generator may further include a lever arm coupled to the support, where the z-axis adjustor may include an actuator. A first carriage may move on a first set of one or more bearings along the support and/or inside the support and may be coupled to the support and the force generator. A second carriage may move on a second set of one or more bearings along the support and/or inside the support and may be coupled to the support and the head support arm. The device may include a recoil element elastically coupling the first carriage and the second carriage such that the force applied by the force generator causes the recoil element to elongate when the pressure is applied to the tissue of the patient and to recoil after the force ceases to be applied by the force generator.

The device may further include a track configured to translate the device along a first axis, referred to as an x-axis, running parallel to a surface that is elongated and usable to receive the patient in a substantially flat position. The fastener may fix the device at a location in the track along the first axis. The device may also include a second hinge coupled to the support and configured to rotate the lever arm around a second axis, referred to as a y-axis, that runs perpendicular to the elongated surface for receiving the patient, the second hinge enabling rotation of the lever arm such that an oblique pressure can be applied from the pressure head to the tissue when the patient is positioned substantially flat on the surface. The device may also include a third hinge coupled to the support and configured to rotate the lever arm around a third axis, referred to as a z-axis, that runs perpendicular to both the y-axis and the z-axis. The third hinge may enable rotation of the lever arm to apply the pressure to the tissue at two or more horizontal locations of the patient without the patient repositioning on the surface.

### BRIEF DESCRIPTION OF THE DRAWINGS

The embodiments of this disclosure are illustrated by way of example and not limitation in the figures of the accompanying drawings, in which like references may indicate similar elements and in which:

FIG. 1A illustrates a treatment device for manipulating a tissue of a patient, a pressure head applying a pressure to the tissue with a multiplied force of a lever arm, one end of the lever arm rotating around a hinge that is elevated by a support above the patient, a practitioner applying force on a grip of the lever arm to generate the pressure and heal the patient, according to one or more embodiments.

FIG. 1B illustrates another treatment device for manipulating the tissue of the patient, including a first track for adjusting grips and/or the pressure head, a head rotation arm for applying a rotational sheer pressure to the tissue of the patient, a fastener mating with a track coupled to a surface on which the patient may be positioned, the track allowing the device to slide along the body of the patient to reposition the pressure head, according to one or more embodiments.

FIG. 2A illustrates another treatment device for manipulating the tissue of the patient, including a head support arm coupled to the pressure head and receiving a force from a force generator, where the head support arm may recoil as a result of a recoil element, for example integrated with the support, according to one or more embodiments.

FIG. 2B illustrates another treatment device for manipulating the tissue of the patient in which the force generator is a lever arm, where the force is imparted from the lever arm to the head support arm through contact between a first contact on the lever arm and a second contact on the head support arm, according to one or more embodiments.

FIG. 3 illustrates yet another treatment device for manipulating the tissue of the patient and further demonstrating a capability to adjust the pressure head to come into contact with the patient at different locations of the body of the patient and at different angles relative to the tissue, including an x axis rotation, a y axis rotation, a z axis rotation, and an x axis translation, according to one or more embodiments.

FIG. 4 illustrates an example embodiment including an adjustable lever arm comprising an internal arm bar and an external arm bar, a hinge bracket and a first hinge point enabling rotation of the lever arm, another hinge bracket and a second hinge point enabling a y axis rotation, a pivot bolt and a pivot plate enabling a z axis rotation, and a track plate and a mounting plate enabling an x axis translation, according to one or more embodiments.

FIG. 5 illustrates another example embodiment in which the treatment device of FIG. 4 further includes an actuator that may be used to automatically apply a force to the pressure head, according to one or more embodiments.

FIG. 6 illustrates an exploded view of the embodiment of the treatment device FIG. 5, according to one or more embodiments.

FIG. 7 illustrates an example integration of a table with the treatment device of FIG. 4 with a table to form a treatment table, including fastening of a mounting track to a frame of the table such that the treatment device may translate almost the full length of the table along an x axis and/or parallel to a vertical axis of the body of the patient to reach various portions of the body of the patient (e.g., from neck to ankle), according to one or more embodiments.

FIG. 8 illustrates a treatment session process flow for utilizing the treatment device, including positioning the patient, selecting an appropriate pressure head, selecting a leverage quantity, configuring axes, applying force to the lever arm to apply pressure to the tissue, and then repeating the process as may be necessary, according to one or more embodiments.

FIG. 9 illustrates a schematic diagram of possible electronic sensor integration and data generation and use, including use of multiple treatment sensors recording data (e.g., pressure, head rotation, temperature) that may be displayed to a practitioner in realtime and/or may be recorded in a session data, and further including a computing device comprising a session database storing one or more session data from a treatment session of the patient, according to one or more embodiments.

FIG. 10 illustrates a data acquisition process flow demonstrating the generation of a session data object and the subsequent gathering of session data to enhance consistency and/or efficiency of present or future treatments, according to one or more embodiments.

FIG. 11 illustrates a patient in a face-up position receiving an example treatment session with the device of FIG. 4 for a shoulder injury, according to one or more embodiments.

FIG. 12 illustrates the patient of FIG. 11 in a face-down position receiving the example treatment for the shoulder injury, according to one or more embodiments.

FIG. 13 illustrates another patient in a face-up position receiving an example treatment session with the device of FIG. 4 for a knee injury, according to one or more embodiments.

FIG. 14 illustrates the patient of FIG. 13 in a face-down position receiving the example treatment for the knee injury, according to one or more embodiments.

FIG. 15 illustrates an example embodiment in which a head support arm may be actuated to apply the pressure to the tissue of the patient by either a force generator such as an off-axis cam operated by a motor and/or a lever arm that may be manually operated by the practitioner, according to one or more embodiments.

Other features of the present embodiments will be apparent from the accompanying drawings and from the detailed description that follows.

### DETAILED DESCRIPTION

Disclosed are a method, a device, and/or system of deep tissue and/or myofascial treatment of a patient 2 by imparting a multiplied force on a pressure head 10 to a tissue of the patient 2. Although the present embodiments have been described with reference to specific example embodiments, it will be evident that various modifications and changes may be made to these embodiments without departing from the broader spirit and scope of the various embodiments.

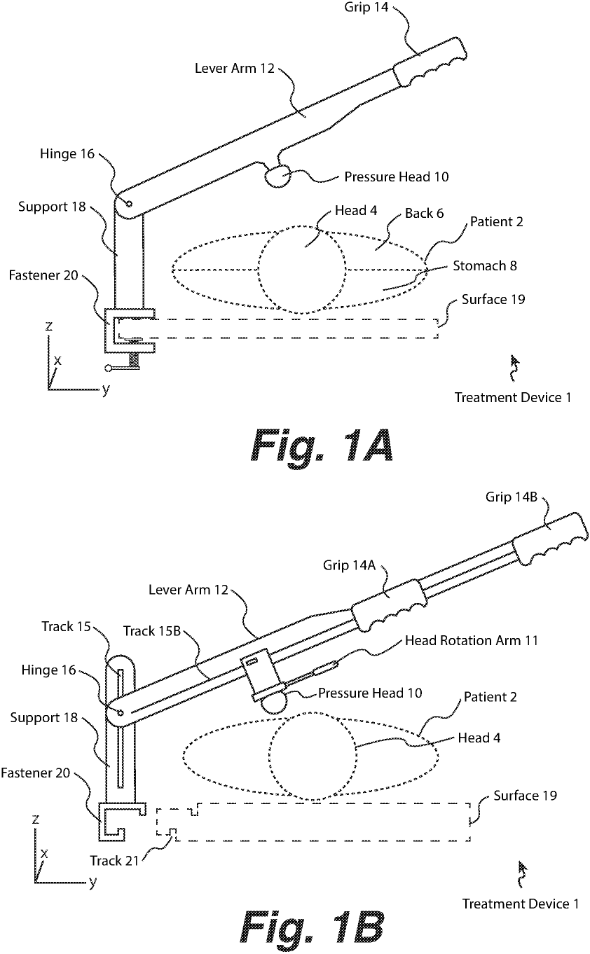

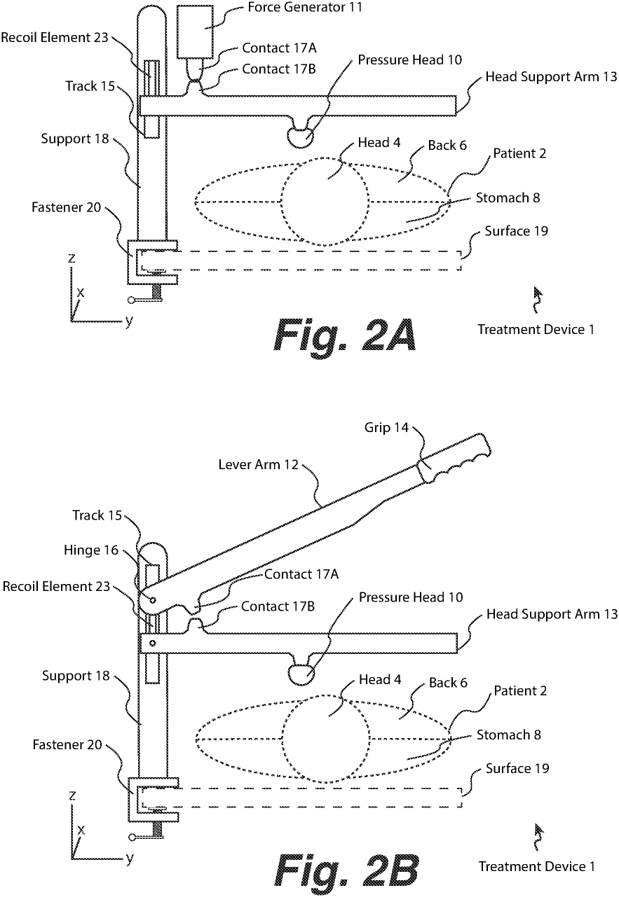

FIG. 1A illustrates a treatment device 1 according to one or more embodiments. The treatment device 1 may be utilized by a practitioner (e.g., a physician, a physical therapist, a masseuse, an acupuncturist, a chiropractor, and/or another type of health or wellness provider) to treat a patient 2. Using expertise of the practitioner and/or other instructions, the practitioner may identify points on the body of the patient 2 requiring manual manipulation and/or pressure, including a tissue of the patient 2. For example, for an injury in a certain area of the body the practitioner may utilize professional knowledge, and sometimes feedback from the patient (e.g., levels of pain, stiffness, lack of mobility, or discomfort) to determined which muscels, joints, and tissues at various depths, should receive manual pressure.

The practitioner may utilize the treatment device 1 to apply pressure to a tissue of the patient 2 at one or more locations. The patient 2 may be positioned relative to the treatment device 1, for example lying flat on a surface 19, as shown in FIG. 1A, and/or with the tissue of the patient 2 lying flat against the surface 19. In one or more embodiments, the patient 2, a portion of the patient 2 (e.g., the back, while the knees are bent), and/or a tissue of the patient 2 may be positioned substantially flat against the surface 19, where substantially means small variations in surface flatness may occur to accommodate the body of the patient 2, for example depressions in a cushion or padding, ergonomic countering for receiving a particular body part, etc. In one or more embodiments, the surface 19 may also be substantially level (e.g., ±10 degrees), for example a surface on a table. The patient may lay on the surface 19 either on a back 6 of the patient 2, on a stomach 8 of the patient 2, on a side of the patient, and/or in another configuration. Specifically, the patient 2 of FIG. 1B is shown lying on their stomach 8 such that the back 6 of the patient 2 is facing toward the pressure head 10 of the treatment device 1.

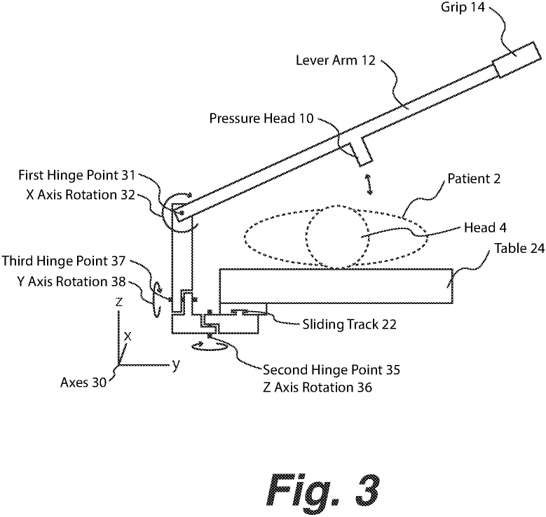

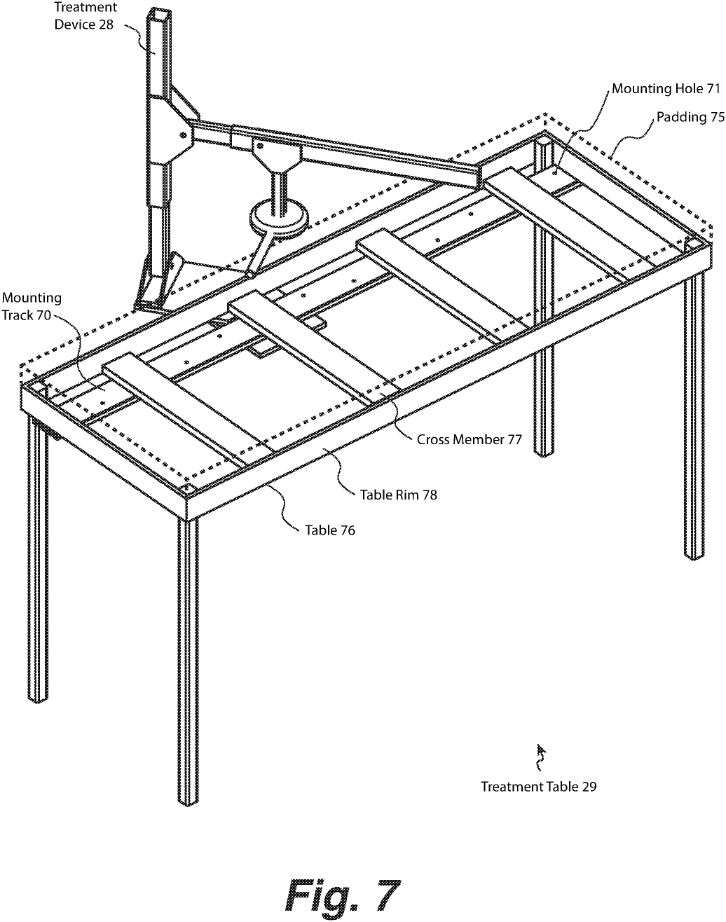

The treatment device 1 may be spatially fixed relative to the patient 2 and/or the surface 19 to which the patient is associated, for example through gravity, friction, adhesion, or another force. In one or more embodiments, and as shown in FIG. 3 and FIG. 7, the surface 19 may be a table. Fastener 20 may be used to attach the treatment device 1 to the surface 19. Specifically, in FIG. 1A and one or more other embodiments, the fastener 20 may comprise a “C” clamp, although other fasteners may also be utilized.

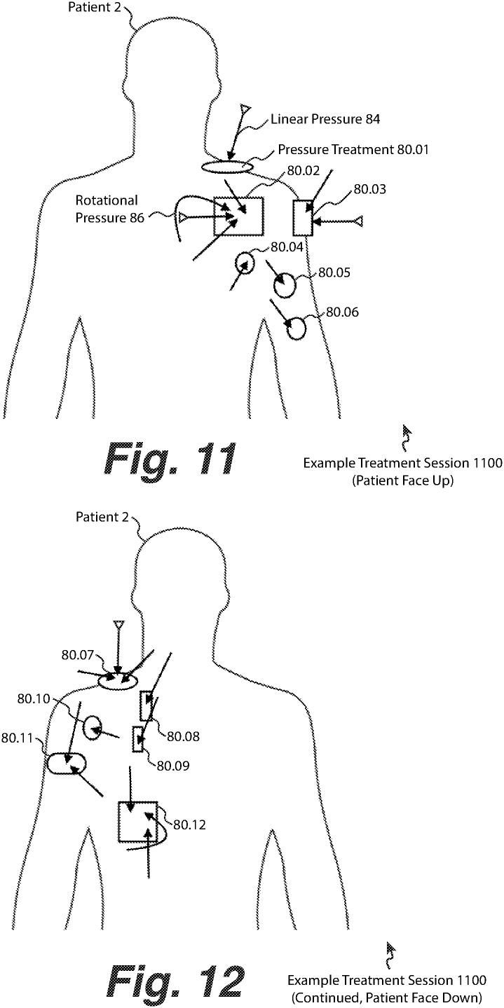

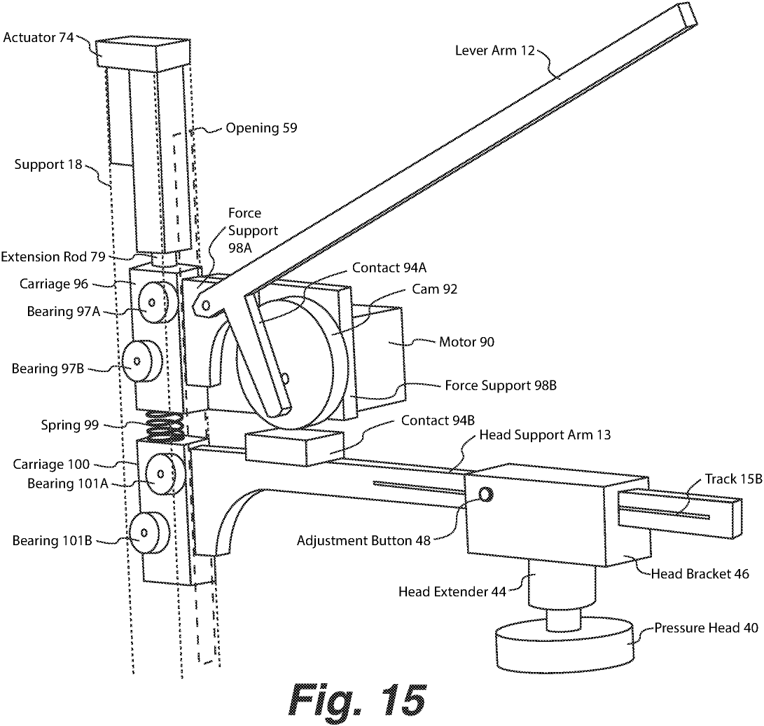

The practitioner may place the pressure head 10 in position on a tissue of the patient 2 by moving a lever arm 12 that rotates around a hinge 16. Alternatively, or in addition to the lever arm 12, and as further shown and described in FIG. 2A, FIG. 2B, and FIG. 15, the pressure head 10 may be attached to a head support arm 13 and moved into position on a the tissue of the patient 2. The hinge 16 may be elevated above the surface 19 by a support 18 to create enough clearance for the patient 2 and/or promote an approximately normal angle of engagement between the pressure head 10 and the tissue of the patient 2 (assuming no changes in axes, as described below). The practitioner may then exert force on a grip 14, the force multiplied by the lever arm 12 at the pressure head 10 (e.g., which may be referred to herein as a “multiplier force”). The application of pressure may be referred to herein as a “pressure treatment” (e.g., the pressure treatment 80 of FIG. 11 though FIG. 14). Multiple pressure treatments may be utilized in a treatment session to work on, adjust, treat, and/or heal the patient 2.

The pressure treatment using the treatment device 1 of FIG. 1A, and one or more other embodiments, may have multiple benefits. First, the practitioner may be able to use less force due to the amplification of the lever arm 12, which may conserve strength and energy of the practitioner so that they can provider longer treatment sessions and/or treat more patients 2 before a significant resting period. Second, a larger pressure may be able to be applied to the tissue of the patient 2 (e.g., 250 psi, 500 psi, 1000 psi). This may be helpful for injuries or muscle tightness of certain types (including deep tissue injury), and also may be useful where the practitioner is smaller in size and/or strength relative to the patient 2. Many other advantages of the treatment device 1 and various features are further shown and described throughout the present embodiments.

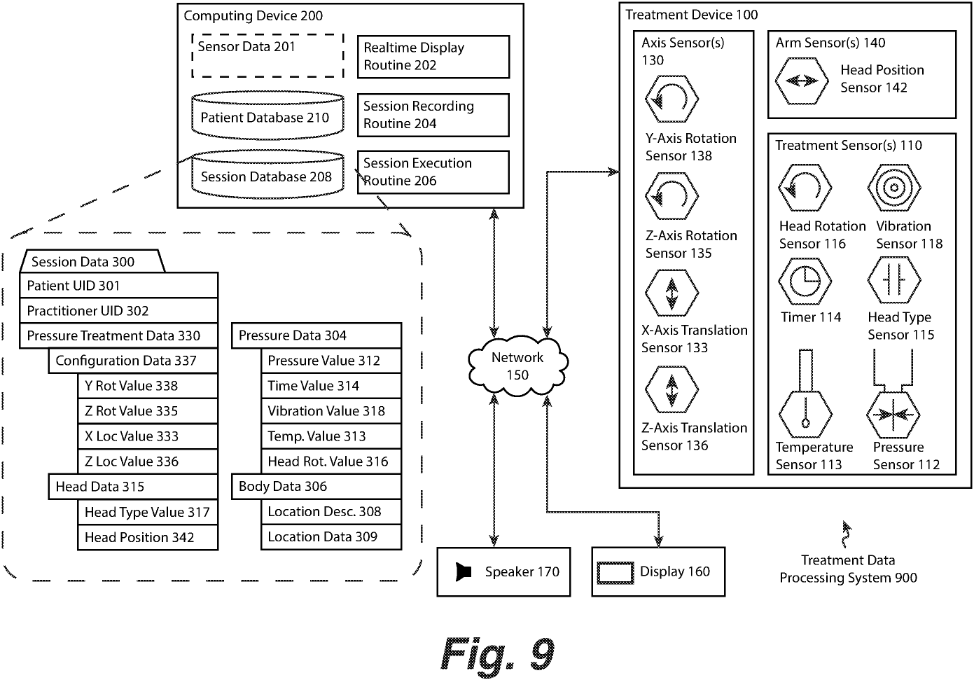

Each pressure treatment may adjust a number of variables to vary its treatment capability and/or treatment profile. For example, as will be described further in FIG. 3, it is possible to adjust an angle of the pressure head 10 relative to a tissue of the patient 2. In one or more embodiments, various pressure heads 10 may be used and/or replaced on the lever arm 12 (and/or the head support arm 13) with different shapes, surface textures, surface frictions (e.g., high friction to prevent sliding on the skin, low friction to slide along the skin), and/or other properties. In one or more embodiments, and as described further in conjunction with the embodiment of FIG. 9, the pressure head 10 and/or the pressure treatment may include vibration, temperature control (e.g., heat and/or cool), and other aspects useful in tissue healing as known in the art. In one or more embodiments, electronic data feedback, recording, and/or retrieval may be used in providing pressure treatments and/or pressure sessions, as shown and described in conjunction with the embodiment of FIG. 9.

The patient 2 may reposition and/or may be repositioned multiple times during the treatment session, and possible multiple related or unrelated bodily areas may be treated. For example, and as shown in detail in the example of FIG. 11 through FIG. 14, the patient 2 may have both a knee injury and a shoulder injury treated in a single treatment session. Each separate area treated within a treatment session may be referred to herein as a “treatment subsession.” The practitioner may also use traditional techniques during a treatment session, such as hand massage, other auxiliary tools, etc.

It should be noted that the patient 2 need not be lying on the surface 19. In one or more embodiments, FIG. 1A may be seen as a top-down version where the surface 19 is a wall or other fixed object, and the patient 2 stands against the wall to receive the pressure treatment. In one or more embodiments, the surface 19 may also include one or more means for calibration that may assist in ensuring the pressure head 10 will record a correct location of engagement with a tissue. A calibration procedure is also possible in which the practitioner “touches” the pressure head 10 to multiple control points, such as head, shoulders, elbows, waist, knees, toes, etc., such that a spatial map of the patient 2 may be stored. Other automatic means of generating a spatial calibration and/or spatial map are possible, such as IR detection, LIDAR sensors, and other devices, systems, and methods known in the art.

FIG. 1A illustrates a method of treatment which may be independent of the treatment device 1. In one or more embodiments, a method for bodily tissue manipulation includes positioning a patient 2 on a surface 19. The method places a pressure head (e.g., the pressure head 10) on a tissue of the patient. The pressure head 10 is coupled to a lever arm (e.g., the lever arm 12) pivoting on a fulcrum that is fixable relative to the surface 19. As just one example, as shown in FIG. 1A, the hinge 16 acts as the fulcrum which is fixed by the support 18 and the fastener 20 to the surface 19. The lever arm 12, as may be known in the art of physics and/or mechanical engineering, may include an effort portion of the lever arm 12, a load portion of the lever arm 12, and the fulcrum. The fulcrum may be the pivot point. A force may be applied to the lever arm 12 at the effort portion of the lever arm 12, for example an end opposite the fulcrum (e.g., the grip 14 in FIGS. 1). The method may then apply a multiplier force to the pressure head 10 at the load portion of the lever arm 12 to provide a pressure treatment for healing the patient 2 (e.g., the pressure treatment 80). Such pressure treatment may be able to be duplicated and/or is repeatable (e.g., due to a known leverage ratio and/or force multiplier), while reducing exertion on a practitioner applying the force to the lever arm 12. It should be noted that while the treatment device 1 illustrates a certain configuration of fulcrum, pivot point, effort portion of a lever arm 12, and load portion of a lever arm 12, other configurations are possible and will be appreciated by one skilled in the art. Additional aspects of the method that may be utilized are described in conjunction with the description of the embodiment of FIG. 3.

FIG. 1B illustrates another example of the treatment device 1 utilizing a lever arm 12, according to one or more embodiments. In the embodiment of FIG. 1B, and one or more other embodiments, the treatment device 1 may include two or more instances of the grip 14 (e.g., a grip 14A, a grip 14B, etc.). Each instance of the grip 14 may be placed in areas of convenience for the practitioner and/or at set leverage and/or force multiplier ratios. In one or more embodiments, a single instance of the grip 14 and/or each instance of the grip 14 may also be adjustable along a length of the lever arm 12, for example sliding along a track 15B and/or otherwise able to be fastened at different locations. As just one example, the grip 14 may be able to be adjusted such that leverage ratios of 1:1, 1:1.5, 1:2, 2:3, and/or 1:3 may be achieved.

In the embodiment of FIG. 1B, and in one or more other embodiments, the treatment device 1 may include a pressure head 10 that is adjustable along the lever arm 12. For example, the pressure head 10 may include a depressible button unlocking its position such that it can slide and re-lock in a different location along the lever arm 12. Repositioning the pressure head 10 may allow the pressure head 10 to reach certain tissues of the patient 2, and/or may also be used to adjust the leverage ratio and/or force multiplier, including in combination with adjustment of the grip 14. In one or more embodiments, adjusting the pressure head 10 along the lever arm 12 may also prompt the practitioner to adjust the grip 14 to maintain a similar and/or the same leverage ratio. In one or more other embodiments, including use of realtime feedback from a pressure sensor 112 as described in conjunction with FIG. 9, readjustment of the pressure head 10 and/or the grip 14 may still easily allow the practitioner to maintain similar pressures (albeit with different force exertion requirements by the practitioner).

In FIG. 1B, and in one or more embodiments, the pressure head 10 may rotate or swivel, for example when the practitioner applies force to a head rotation arm 11. The rotation of the pressure head 10 when in contact with the tissue of the patient 2 may enable application of a rotational pressure (e.g., the rotational pressure 86), especially where the pressure head 10 is a high-friction polymeric material placed directly against the skin of the patient 2. In one or more embodiments, this rotational pressure may further assist in loosening muscles, massaging deep tissue, unwinding sarcomere knots, and effecting other healing functions. The rotation utilized in such a pressure treatment may be relatively small, for example ±15 degrees of rotation, or ±60 degrees of rotation. Numerous rotational mechanisms may be used for the pressure head 10. The pressure head 10 may rotate freely (e.g., on a ball bearing swivel or taper bearing), or may be brought back to a central resting position by a spring or other mechanism. Although a manual head rotation arm 11 is shown, other methods of providing the rotation will be appreciated, including a motor-driven pressure head 10.

In FIG. 1B, and in one or more other embodiments, a track 15 may be utilized to allow one end of the lever arm 12 to change its position relative to the surface 19. For example, a fastener may be used to tighten and/or fix the lever arm 12 at a location in the track 15. The fastener may or may not be related to a mechanism of the hinge 16. The track 15 may make it easier for the practitioner to treat different body types, girths, and/or body configurations of the patient 2.

In FIG. 1B, and one or more embodiments, the fastener 20 may be a set of rails that slide on a track 21. This may allow the treatment device 1 to slide along the surface 19 in what is arbitrarily defined herein as along an x axis. This may allow repositioning of the treatment device 1 such that, for example, a first pressure treatment may be applied to the neck of the patient 2 and a second pressure treatment may be applied to a calf of the patient 2, where between the first pressure treatment and the second pressure treatment the treatment device 1 was translated along the track 21 effecting an x axis translation. An example of the track 21 is further shown and described in conjunction with the embodiment of FIG. 3 through FIG. 7.

FIG. 2A illustrates another example of the treatment device 1 for manipulating the tissue of the patient 2, including a head support arm 13 coupled to the pressure head 10 and receiving a force from a force generator 11, according to one or more embodiments. The force generator 11 may be and/or may comprise, for example, an actuator, a pneumatic piston, a lever (e.g., which may be similar or identical to the lever arm 12 or may be powered or motor operated), a controllable weight, a pully system, a gear system (e.g., such as a worm drive), a cam (e.g., the cam 92 as shown in FIG. 15), and/or other suitable devices, systems, and/or methods known in the art for generating usable force. Optionally, the force may be imparted from one contact point to another, e.g., from a contact 17A of the force generator 11 to a contact 17B of the head support arm 13. The contact points may include special hard and/or low-friction materials to prevent sound, allow for low friction sliding (e.g., of a cam), etc.

The force generator 11 may be fixed relative to the surface 19 and/or the support 18. As just one example, and although not shown in FIG. 2A, the force generator 11 may be coupled to the support 18, for example as shown in FIG. 2B (where the force generator 11 is the lever arm 12) and FIG. 15 (e.g., where the force generator 11 comprises the lever arm 12 and the cam 92 and motor 90).

When the force is applied to the head support arm 13, the head support arm 13 may move and/or slide along the support 18, or otherwise move toward the patient 2 until the pressure head 10 has applied the pressure treatment to the tissue of the patient 2. As shown, the movement when the force is applied to the head support arm 13 may occur along a z axis that runs parallel to the support 18. As further described in conjunction with the embodiment of FIG. 3, the z axis may be referred to as the axis 30z and may be parallel to a frontal axis of the patient 2.

Following application of the force by the force generator 11, the head support arm 13 may be moved in an opposite direction to bring about the cessation of the pressure treatment. A recoil element 23 may be a mechanical device capable of generating recoil and/or rebound in the head support arm 13. For example, the recoil element 23 may be a coil spring, a leaf spring, a counterbalance, and/or an elastic material. In one or more embodiments, the force generator 11 may be primarily capable of generating a force along a single vector (e.g., a negative z axis direction), where the recoil element 23 may then reposition the head support arm 13 and the pressure head 10 such that the pressure treatment is arrested. In one or more embodiments, the head support arm 13 may be fixed in a track 15 and the head support arm 13 may be moved by the force, remaining substantially perpendicular (e.g., ±5 degrees) to the surface 19 when the force is applied. The recoil element 23 may then return the head support arm 13 to its original position. Although the recoil element 23 is shown above the head support arm 13 and inside a cavity of the support 18, in one or more embodiments the recoil element 23 may be placed below the head support arm 13 (e.g., subject to compression during application of the force on the head support arm 13) and/or in another suitable configuration to generate desired recoil.

In one or more embodiments, and as further described in FIG. 5, a z axis adjustor such as an actuator 74 and/or other methods of z-axis adjustment may be able to effect pre-treatment positioning for the z axis, for example to accommodate a girth of the patient 2 or reposition the treatment device 1 for various body parts. This function may be different than a subsequent z axis movement for the purpose of applying the pressure treatment. Separate function for initial z axis adjustment and z axis movement for treatment is further illustrated in FIG. 15 in which both the actuator 74 and a spring 99 may allow for variation in the z axis (e.g., to provide for pre-treatment adjustment and to apply the pressure treatment, respectively). In one or more other embodiments, however, the same mechanism may be used for both initial z axis adjustment and z axis movement of the pressure head 10 to apply a pressure treatment.

A pressure treatment resulting from the treatment device 1 of FIG. 2A, and one or more other embodiments, may have multiple benefits. First, where the force generator 11 is a motor, piston, or other powered system or device, the practitioner may generate a force that is automatically imparted to the pressure head 10 by the force generator 11, which may conserve strength and energy of the practitioner so that they can provider longer treatment sessions and/or treat more patients 2 before a significant resting period. Second, a larger pressure may be able to be applied to the tissue of the patient 2 (e.g., 250 psi, 500 psi, 1000 psi). Third, automated and powered pressure treatments may be able to be more consistant.

FIG. 2A illustrates a method of treatment which may be independent of the treatment device 1. In one or more embodiments, a method for bodily tissue manipulation includes positioning a patient 2 on a surface 19. The method places a pressure head (e.g., the pressure head 10) on a tissue of the patient. The pressure head 10 is coupled to support member (e.g., the head support arm 13) moving along a z-axis that is fixable relative to the surface 19. As just one example, as shown in FIG. 2A, the head support arm 13 may be substantially perpendicular to the support 18 and the fastener 20 may be fastened to the surface 19. The head support arm 13 may include a load portion which may receive a force (e.g., at the contact 17B). The method may then apply the force to the pressure head 10 to provide a pressure treatment for healing the patient 2 (e.g., the pressure treatment 80). Such pressure treatment may be able to be duplicated and/or is repeatable repeatable (e.g., due to known and/or defined power provided to the force generator 11), while reducing exertion on a practitioner. It should be noted that while the treatment device 1 of FIG. 2A illustrates a certain configuration of the lever support arm 13, other configurations are possible and will be appreciated by one skilled in the art.

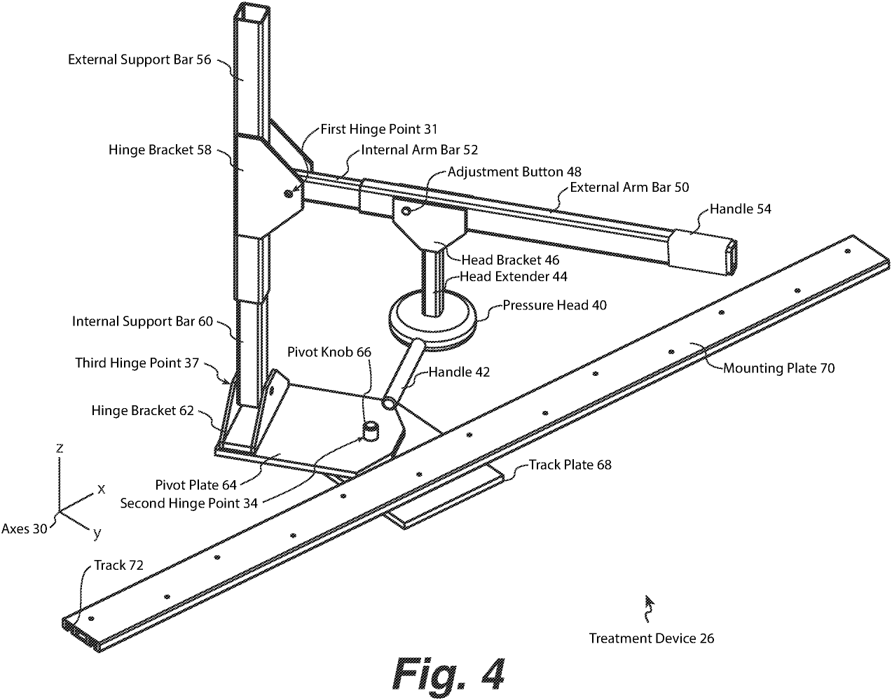

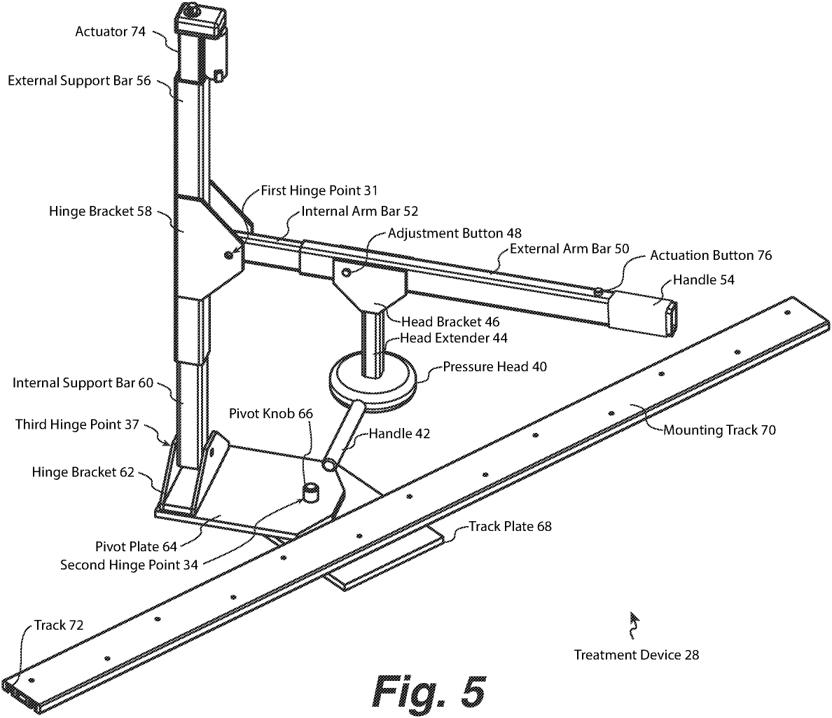

FIG. 2B illustrates another example instance of the pressure treatment device 1 utilizing the head support arm 13 in which the force generator 11 is the lever arm 12. The contact 17A of the lever arm 12 may apply the force to the contact 17B to move the head support arm 13 along the z axis to apply the pressure treatment. Both the lever arm 12 and the head support arm 13 may slide within the track 15, for example until the practitioner fixes a location of the lever arm 12. The recoil element 23 may couple the lever arm 12 to the head support arm 13 such that when the lever arm 12 rotates around the hinge 16 and applies force to the head support arm 13 (e.g., via the contact 17A and the contact 17B) the recoil element 23 may lengthen to allow the downward (e.g., negative z axis) movement of the head support arm 13 to apply the pressure treatment. Another example of the force generator 11 and/or the lever arm 12 being connected to the head support arm 13 through a recoil element 23 is illustrated in FIG. 15. In addition, it will be apparent that the embodiments of FIG. 2A and FIG. 2B may include other features of the present embodiments, for example an adjustable grip similar to FIG. 1B, a head rotation arm 11, a sliding pressure head 10 adjustable along the y axis (e.g., as shown in FIG. 4 through FIG. 6), etc. The contact 17A and/or the contact 17B may include a low-friction surface and/or a wheel. For example, the contact 17B may be omitted and the contact 17A may include a wheel such that during depression of the lever arm 12 the wheel may roll along an upper surface of the head support arm 13. Although not shown, it should be noted that a contact point of the contact 17A may be adjusted along the lever arm 12 and/or the contact point of the contact 17B may be adjusted along the head support arm 13, for example similarly to adjustment of the pressure head 10 on the track 15B of FIG. 1B.

FIG. 3 illustrates yet another example of the treatment device 1, according to one or more embodiments. In one or more embodiments, and the embodiment of FIG. 3, the treatment device 1 may be adjustable such that pressure head 10 may engage with the patient 2 at various body locations and various angles. The angle of engagement with the tissue of the patient 2 may assist in treatment, for example by providing “sheer pressure.”

In one or more embodiments, a set of axes 30 may be arbitrarily assigned to the surface 19, or in the case of FIG. 3 the table 24. An origin of the set of axes 30 may be arbitrarily defined to have a known association to a point on the surface 19 and/or a point on the body of the patient 2 (e.g., the crown of the head). In one or more embodiments, an x axis (which may be referred to as the axis 30x) may be defined as running parallel a length of the table 24, a y axis (which may be referred to as the axis 30y) is perpendicular to the axis 30x and defined as running parallel to a width of the table 24, and a z axis (which will be referred to as the axis 30z) is perpendicular to the axis 30x and the axis 30y, and may run perpendicular to the surface of the table 24.

In one or more embodiments, the treatment device 1 may include a first hinge point 31 that may effect an x axis rotation 32. Primarily, this may provide the pivot point of the fulcrum of the lever arm 12. A second hinge point 35 may enable a z axis rotation 36 which is a rotation around the z axis 30z. The z axis rotation 36 may be used, for example, to reposition the pressure head 10 relative to a tissue of the patient 2 and/or to provide a different angle of engagement with the tissue and/or to generate a sheer pressure. A third hinge point 37 may enable a y axis rotation 38, which may also enable repositioning of the pressure head 10 relative to a tissue of the patient 2 and/or to provide a different angle of engagement with the tissue and/or generate a sheer pressure. The third hinge point 37 may allow the lever arm 12 (and/or the head support arm 13) to “tip over” to a different angle. Any of the hinge points, and especially the hinge point 35 and the third hinge point 37, may include several positions in which an axis rotation may be locked (e.g., each 5 degrees, each 15 degrees, etc.). In one or more embodiments, and as previously described, a sliding track 22 (e.g., an instance of the track 21) may be used to effect an x axis translation.

It should be noted that other translations along the axes 30 are possible. For example, the treatment device 1 may adjust relative to the surface in a y axis translation, where a second instance of a sliding track (not shown) parallel to sliding track 22 may translate the treatment device 1 away from or toward an edge of the table 24. Other mechanisms to effect a y axis translation may include a hydraulic cylinder. A y axis translation may, for example, allow for repositioning of the pressure head 10 across the width of the patient 2 (e.g., along a frontal axis of the body of the patient 2, as such bodily axis is known in the art) without an adjustment pressure head 10 along the lever arm 12 (and/or the head support arm 13) and/or repositioning of the patient 2 on the table 24. Similarly, a track, hydraulic lift, and/or other mechanism may be utilized to effect a z axis translation, which may have a similar effect as extending or retracting the support 18. This may be helpful to accommodate patients 2 of different sizes, shapes, or placed in various spatial configurations. In one or more embodiments, it should be noted that the treatment device 1 may be a freestanding device attached or affixed to a floor, where the table 24 is also freestanding. In such case, a z axis translation may be effecting through a jack or hydraulic lift mechanism, and/or raising or lowering the table 24.

In one or more embodiments, the lever arm 12 (and/or the head support arm 13) may be counterweighted with springs, weights, or other mechanisms such that heavier attachments may be added to the pressure head 40 and/or such that the lever arm 12 may appear lighter to the practitioner. Counterweighting may be configurable and/or adjustable, depending on the strength of the practitioner or the weight of the lever arm 12, pressure head 10, and/or other attachments.

The axes 30 and/or its origin point in space may be arbitrarily defined relative to the treatment device 1, the surface 19, and/or the patient 2. In one or more embodiments, repositioning of the pressure head 10 to perform a pressure treatment (e.g., a pressure treatment 80) may be completed relative to axes aligned with the patient 2. As known in the art, a human body such as that of the patient 2 may have a vertical axis, a frontal axis, and a sagittal axis. Referring to FIG. 3, in one or more embodiments the vertical axis may be going through the crown of the head 4 and out between the arches of both feet (e.g., “into and out of the page”), the frontal axis may extend through a point somewhere on the stomach and back of the patient 2, and the sagittal axis may extend through a point somewhere on the left side abdomen and the right side abdomen of the patient. In one or more embodiments, where the patient 2 is lying on an elongated instance of the surface 19 such as the table 24, the x axis 30x may be defined as parallel to the vertical axis of the patient 2, the z axis 30z may be parallel to the frontal axis of the patient 2, and the y axis 30y may be parallel to the sagittal axis of the patient 2.

Referring to the above method, in one or more embodiments, the lever arm 12 (and/or the head support arm 13) and/or the pressure head 10 may be translated parallel to the vertical axis of the patient 2 and the pressure head 10 may be immovably fixed relative to the surface 19. For example, the lever arm 12 (and/or the head support arm 13) may be mounted on a wall, while still effectively providing pressure to a nearby and freestanding instance of the table 24 on which the patient 2 is positioned. The pivot point of the fulcrum may be rotated around an axis perpendicular to the frontal axis of the patient 2, wherein the force results in an oblique pressure applied by the pressure head 10. For example, where the patient 2 is lying on their back or stomach, such rotation may be used to generate a sheering pressure (e.g., a sheering pressure 86) that may be at an angle that is not normal to the target tissue of the patient 2. The method may also rotate the pivot point of the fulcrum around an axis perpendicular to a sagittal axis of the patient 2 to reposition a contact point of the pressure head 10 to the tissue of the patient 2. The force multiplier may be changed by adjusting a length of the effort portion of the lever arm, a position of the pressure head 10 along the lever arm 12, and/or a position of the grip 14 along the lever arm 12. The method may also record a parameter of the pressure treatment (e.g., the pressure treatment 80) dependent on at least one of: the length of the effort portion of the lever arm 12, the position of the pressure head 10 along the lever arm 12, the position of the grip along the lever arm 12, the force applied to the lever arm 12, the force applied by the pressure head 10, a location along the axis parallel to the vertical axis of the patient 2, a rotation around the axis perpendicular to the frontal axis of the patient 2, and/or a rotation around the axis perpendicular to a sagittal axis of the patient 2. As further described in conjunction with the embodiment of FIG. 9, the method may also record a time over which the pressure is applied, a rotational sheer pressure, a vibration setting, and/or a type of pressure head 10.

FIG. 4 illustrates a specific example of the treatment device 1, referred to as the treatment device 26, according to one or more embodiments. A lever arm (e.g., the lever arm 12) may be comprised of an internal arm bar 52 and an external arm bar 50, where the external arm bar 50 adjustably telescopes on the internal arm bar 52. The internal arm bar 52 may be attached at one end to a first hinge point 31, and the external arm bar 50 may include a handle 54. The internal arm bar 52 and the external arm bar 50 may be made of steel, aluminum, carbon fiber, fiberglass, polymer, or another material of sufficient strength to support the pressure head 40 and withstand a force applied to the handle 54. For example, the internal arm bar 52 and the external arm bar 50 may be made from hollow extruded aluminum tubing with a rectangular cross section. Other cross section shapes are also possible. The handle 54 may be, for example, a wrap comprising a plastic, a fabric, or leather, that is sewn, glued, friction fit, and/or otherwise affixed or mounted to the external arm bar 50.

A pressure head 40 (e.g., an instance of the pressure head 10) may be coupled to the external arm bar 50 with a head bracket 46 and a head extender 44. In one or more embodiments, the head bracket 46 may slide along a track on the external arm bar 50, where a depressible adjustment button 48 may be used to free or fix the head bracket 46 in infinitely variable and/or fixed locations along the external arm bar 50. Alternatively, the telescoping lever arm 12 may be used to adjust the location of the pressure head 40 along the frontal axis of the patient 2. The head bracket 46 may be welded, bolted, or otherwise affixed to the head extender 44, and the head extender 44 may be rotatably mounted to the pressure head 40 through a pivot or swivel. The pressure head 40 may be replaceable, for example attaching to the head extender 44 with a bolt, a quick-release ball-bearing mechanism, a friction fit, a spline fitting, a rail system, and/or another device or method. The handle 42 may be used to provide rotational pressure (e.g., the rotational pressure 86) when the pressure head 40 is placed in contact with a tissue of the patient 2. In one or more embodiments, the entire pressure head 40 and handle 42 may be made from a single material, such as plastic. In one or more other embodiments, the pressure head 40 may be made of metal or other structural materials but include replaceable elements of other materials for contact with the patient 2 (e.g., friction pads, silicone or urethane blocks replaceable on the contact surface (not shown), etc.).

The first hinge point 31 may be implemented with a hinge, including for example a circular rod extending through the hinge bracket 58 and the internal arm bar 52. For example, the rod may be a bolt, or ball lock pin. A support for the hinge bracket 58 (e.g., the support 18 of FIGS. 1) may include an external support bar 56 telescoping on an internal support bar 60. The hinge bracket 58 may be bolted, welded, or otherwise affixed to the external support bar 56. The internal support bar 60 and the external support bar 56 may be made of steel, aluminum, carbon fiber, fiberglass, polymer, or another sufficiently strong material to support the lever arm (e.g., the lever arm 12) and withstand a force applied to the handle 54. For example, the internal support bar 60 and the external support bar 56 may be made from hollow extruded steel tubing with a rectangular cross section. Other cross section shapes are also possible. The internal support bar 60 may be coupled to a third hinge point 37, for example implemented by a hinge such as a rod or bolt. The third hinge point 37 may have mediated angles allowing for fixed rotations, for example allowing the support 18 to tip and lock into five increments, or, for example, common angles such as fifteen degrees, thirty degrees, and forty-five degrees. A locking mechanism is not shown in the embodiment of FIG. 4, but many will be familiar to one skilled in the art.

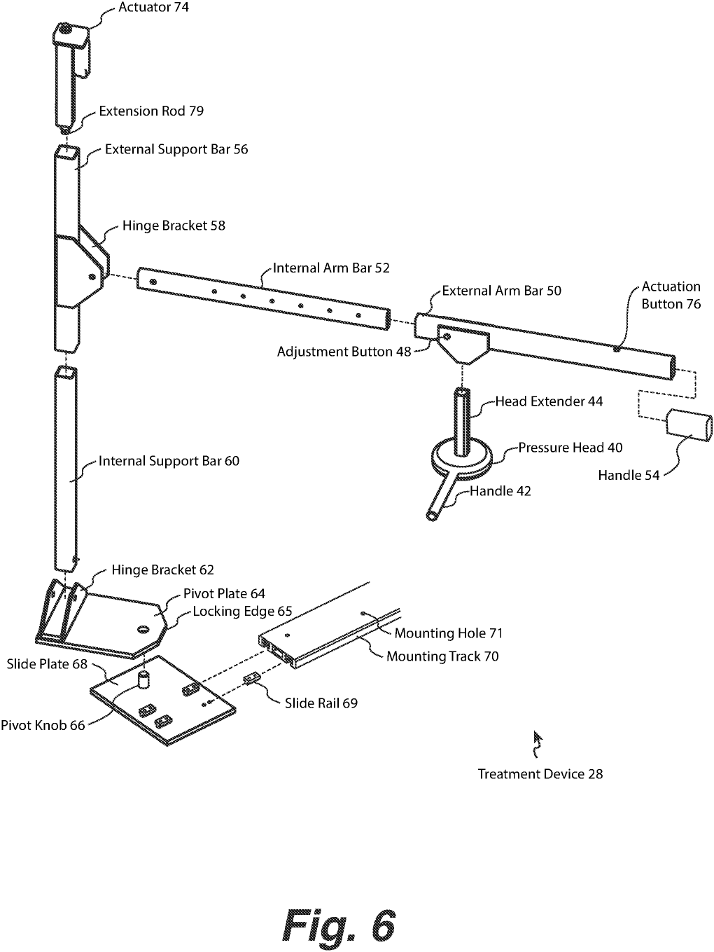

The hinge bracket 62 may be coupled to a pivot plate 64, for example welded or bolted. A second hinge point 34 may enable a z axis rotation 36 (e.g., a rotation around the z axis 30z). The second hinge point 34 may be effected through a hole in the pivot plate 64 through which a pivot knob 66 coupled to and/or producing from a track plate 68 may extend. Two or more angled ridges in the pivot plate 64 may come in contact with a mounting plate 70 to fix the pivot plate 64, and therefore the lever arm 12, into two or more instances of a z axis rotation 36. Specifically, in the embodiment of FIG. 4, a partial octagonal interface is defined on an end of the pivot plate 64 opposite the hinge bracket 62, enabling five rotational positions of a rotation around the z axis 30z at zero degrees, forty five degrees, ninety degrees, negative forty five degrees, and negative ninety degrees. To change the z axis rotation 36, the practitioner may lift the pivot plate 64 until it is above the mounting plate 70, and then replace it in the pivot plate 64 (as further shown in FIG. 6) on the pivot knob 66 in a different orientation. Alternatively or in addition, a mechanism may be used to separate the pivot plate 64 and the mounting plate 70, such that it can rotate freely around the pivot knob 64 or another type of hinge mechanism, e.g., a tension release handle. Although one mechanism for the second hinge point 34 are shown, many others are possible as will be familiar to one skilled in the art. The track plate 68 includes one or more slide rails 69 (e.g., as shown in FIG. 6) that may slide on tracks 72 inside the mounting plate 70 to effect an x axis translation, for example along the length of a massage table as further shown and described in FIG. 7. The mounting plate 70 may be made of, for example, extruded aluminum interfacing with aluminum or steel slide rails 69 attached to the track plate 68. The position along the track 72 may be lockable, either at any location along the track 72 and/or one or more fixed locations (e.g., each inch, every four centimeters). A sled mechanism which slides along ball bearings inside the mounting plate 70 may be also or alternatively utilized, or other suitable translation mechanisms as known in the art.

FIG. 5 illustrates another specific example of the treatment device 1, referred to as the treatment device 28, according to one or more embodiments. In one or more embodiments, and the embodiment of FIG. 5, the adjustment of the pressure head and/or depression of the pressure head 40 may be power driven and/or power assisted. An actuator 74 (e.g., a pneumatic actuator, a hydraulic actuator) may be able to apply force to the external support bar 56, pushing it down (e.g., effectively shortening the support 18) to provide the pressure to the patient 2. The actuator 74 is further shown and described in conjunction with the embodiment of FIG. 6. The actuator 74 may be powered by a power source, such as 110 V outlet or 12 V battery, and controlled by the practitioner with an actuation button 76 which may apply pressure as long as the actuation button 76 is depressed. More complex control interfaces are possible, for example buttons which initiate movement of the actuator 74 up or down, buttons or switches which set pressure limits, etc. Although one example of a power driven and/or power assisted mechanism is illustrated, it will be apparent to one skilled in the art that many mechanisms are possible, including the power-drive application of force (e.g., use of a force generator 11) to the lever arm 12 and/or the handle 54.

Alternatively, or in addition to the actuator 74, other techniques, methods, and/or mechanisms of applying force to the pressure head 10 may be utilized. In one or more embodiments, an electric motor, a pneumatic, and/or a hydraulic system may be used to depress the lever arm 12 and/or pressure head 10, including in possible combination with a head support arm 13. In one or more embodiments, a motor may be utilized to rotate a cylinder with an offset fulcrum that may rotate 360 degrees, which may be a full stroke down and up. The offset fulcrum can replace the lever arm 12 completely, which may define an instance of the treatment device 1 that may be usable hand-free, for example as shown in FIG. 15. The offset fulcrum (e.g., the offset cam 92 of FIG. 15) and/or crank may be adjustable such that it could be set to turn one full revolution and/or part of a revolution, where the further the fulcrum moves the longer stroke, and vise-versa.

It should be noted that in one or more embodiments the head support arm 13 and the lever arm 12 may be the same arm. For example, a lever arm 12 may be fixable (e.g., via a pin or other mechanism) such that it can remain perpendicular to the support 18 and receive the pressure from a force generator 11 (e.g., that may be an automatic and/or powered application of force, for example from a motor or pneumatic). However, the lever arm 12 may also be able to be unfixed (e.g., by removing the pin) and rotate freely around a hinge point (e.g., the hinge 16, the first hinge point 31) to act as an independent instance of supplying the force to the pressure head 10. The practitioner may be able to rapidly switch between modes depending on the needs of the treatment.

FIG. 6 illustrates an exploded view of the treatment device 28 of FIG. 5, and many of the components of the treatment device 26 of FIG. 4, according to one or more embodiments.

Several holes along the internal arm bar 52 may allow for adjustment of the length of the lever arm 12 by adjusting the length of the external arm bar 50 and the internal arm bar 52 relative to one another. Wiring and/or a controller for the actuator 74 may be added, but for clarity is not shown. Control wiring for the actuation button 76 may run through the hollow interior of the external arm bar 50, the internal arm bar 52, and along the side of the external support bar 56 and/or up through the interior of the external support bar 56 to the actuator 74. Similarly, any power wiring for the actuator 74 may be run along the side of the support 18, and/or through the external support bar 56, and/or through the internal support bar 60 until departing for the power supply. Data wiring for transmitting data, as further described in conjunction with FIG. 9, may run from one or more sensors in the pressure head 40 through the head extender 44 into the interior or along the side of the lever arm 12 (e.g., through the external arm bar 50 and the internal arm bar 52), and along the side or down through the support 18 (e.g., the external support bar 56 and/or the internal support bar 60) where it may depart for a data processing device (e.g., a computer, microcontroller, or other data processing unit). Alternatively, or in addition, data may be wirelessly communicated via Bluetooth® or another technique known in the art.

The track plate 68 may include one or more slide rails 69 mating with one or more tracks 72 of the mounting plate 70. Specifically, in one or more embodiments and the embodiment of FIG. 6, four instances of the slide rail 69 may be coupled to the track plate 68 in two pairs, each pair in linear succession such that each pair slides into one of two tracks 72 of the mounting plate 70. The slide rails 69 may be attached to the track plate 68 with screws, bolts, and/or other fasteners, or may be manufactured as part or, and integrated with, the track plate 68. Only a portion of the mounting plate 70 is shown for clarity. The mounting plate 70 may include a number of fasteners for coupling the mounting plate 70 such that it is fixed relative to the patient 2 (e.g., fixed to a surface 19, to a table 24, to a wall against which the patient stands, etc.). In one or more embodiments, the mounting plate 70 may include one or more mounting holes 71 for attachment of the mounting plate 70 to a surface 19 or table 24 with screws, bolts, or other fasteners.

In one or more embodiments, the track plate 68 may be powered to move along the tracks 72. For example, a worm drive, belt drive, or other motor may be used to translate the track plate 68 (e.g., along with the entire treatment device 1) along the x axis 30x in a positive or negative direction along the surface 19 such as the table 24 and/or the table 76.

In one or more embodiments, a treatment device includes a table (e.g., the table 24) for receiving a patient 2, and a lever arm (e.g., the lever arm 12) fixable relative to the table for multiplying a force exerted on the lever arm at a pressure head (e.g., the pressure head 10). The lever arm can be translated along at least one axis (e.g., the x axis 30x) and rotated around at least one axis (e.g., the first hinge point 31). FIG. 7 illustrates an example of such a treatment device implemented with the embodiment of FIG. 4.

FIG. 7 illustrates a treatment table 29, according to one or more embodiments. The treatment table 29 comprises a table 76 coupled with the treatment device 28 of FIG. 4. For clarity, a surface of the table (which may include a padding 75) is shown in transparency with broken lines in FIG. 7. The mounting plate 70 may be mounted to cross members 77 of the table 76 and/or table rim 78. Screws and/or bolts may extend through one or more of the mounting holes 71 aligned with the cross members 77.

It should be noted that while a rectangular instance of the table 76 is illustrated, many possible surface or table shapes, sizes, and types may be utilized. For example, the table 76 may be a classic massage table, an inpatient hospital bed, a folding massage table capable of various ergonomic configurations, and/or even a dining room table. The table 76 may include, for example, a head space comprising a cavity and a hole for receiving a head of the patient when the patient 2 is positioned on their stomach on the table and/or surface. A set of axes such as the axes 30 may be defined relative to a surface of the table 76. There may be one or more holes in the padding 75 to allow for easier breathing of the patient 2, cradling the head 4 of the patient 2 to reduce movement, and/or permitting the head 4 of the patient 2 to be positioned straight when the patient 2 lies on their stomach 8 on the table 76.

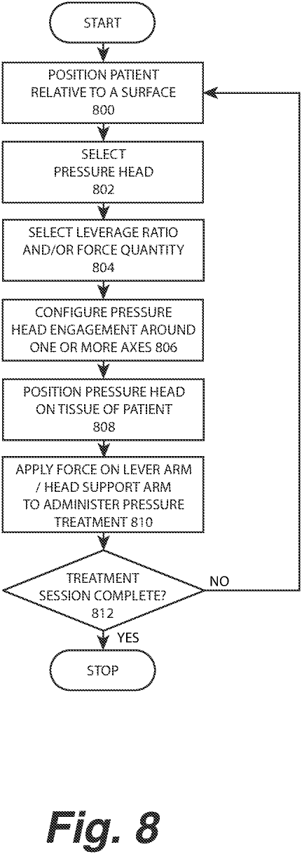

FIG. 8 illustrates a treatment process flow, according to one or more embodiments. Operation 800 positions a patient 2 relative to a surface (e.g., the surface 19). For example, the surface 19 may be a wall, a table, a floor, a bench, and/or an oblique surface that may be at an angle relative to a wall or floor. Operation 802 selects a pressure head 10. Depending on the treatment goals or pressure needs identified by the practitioner, the pressure head 10 may be selected for its mass, coefficient of friction with clothing or bare skin, shape, material, shore hardness, and/or other properties. In addition, certain instances of the pressure head 10 may include capabilities to supply heat, infrared radiation, cool, vibration, ultrasound, and/or other therapeutic modes. Operation 804 may select a leverage ratio and/or force quantity. For example, the practitioner may adjust the pressure head 10, the length of the lever arm 12, and/or the location of a grip 14 to create a certain leverage ratio, force multiplier, and/or force quantity. Operation 806 then may configure the pressure head 10 engagement around one or more axes (e.g., the axes 30), for example to provide an angled and/or sheering pressure. For example, the practitioner may set an x axis rotation 32, a z axis rotation 36, and/or a y axis rotation 38. Alternatively or in addition, the practitioner may effect one or more axis transitions. It is also possible that no axes changes may be selected in operation 806 such that the pressure head 10 may engage with the patient 2 substantially normal to the tissue targeted by the patient 2.

Operation 808 positions the pressure head 10 on a tissue of the patient 2. If the placement appears correct, the practitioner may proceed to operation 810. Otherwise, the practitioner may return to operation 806 to re-adjust the patient 2. The practitioner may also collect initial feedback from the patient 2 in operation 808, such as asking the patient 2 if the contact point feels like it is in the “right” or “correct” location (e.g., a point of pain or tension). Operation 810 then applies force on the lever arm 12 and/or the head support arm 13 to administer a pressure treatment to the patient 2. The force may be applied to the grip 14 by the practitioner, and/or by other means (e.g., a weight and/or a powered force, such as from the actuator 74). The pressure treatment may also comprise a number of other elements, such as heat, cold, vibration, ultrasound, and/or other therapeutic capabilities that may be imparted to the patient through the pressure head 10.

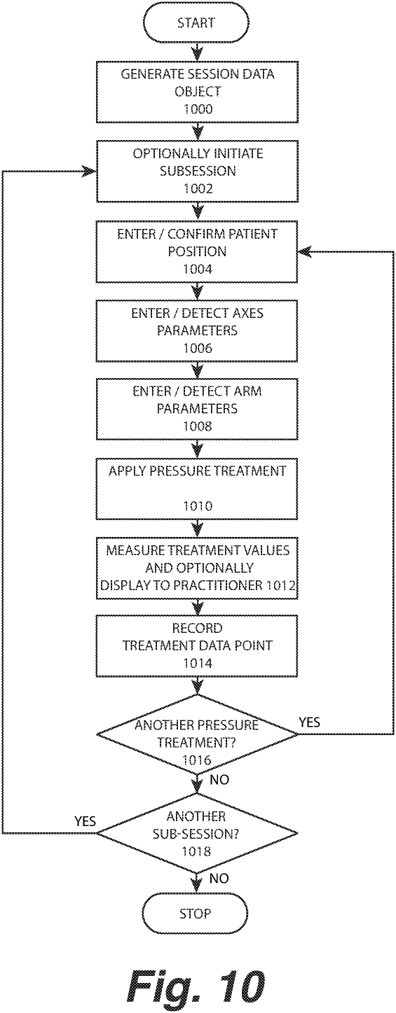

In operation 812, it is determined if the treatment session is complete. If the treatment session is not complete, operation 812 returns to operation 800. Otherwise, operation 812 may end. As further described in conjunction with FIG. 10, a treatment session may also be separated into subsessions, each of which may have its own goal and may be comprised of one or more pressure treatments.

FIG. 9 illustrates a treatment data processing system 900, according to one or more embodiments. In one or more embodiments and the embodiment of FIG. 9, treatment data may be gathered and used in realtime and/or stored to enhance the capability of massage, physical therapy, injury recovery, and/or the application of manual pressure treatments, including without limitation use of the treatment device 1. In one or more embodiments and the embodiment of FIG. 9, a treatment data processing system 900 includes a treatment device 100 (e.g., an instance of the treatment device 1) that may include one or more sensors, several examples of which are illustrated. The treatment device 100 may include a set of one or more treatment sensors 110. The treatment sensors 110 may include a pressure sensor 112, a temperature sensor 113, a timer 114, a head type sensor 115, a head rotation sensor 116, and/or a vibration sensor 118.

The pressure sensor 112 may sense a pressure applied to one or more points of the pressure head 10. Alternatively, or in addition, an intermediate location indicative of pressure may be utilized (e.g., a point in the head extender 44). The pressure sensor may generate a pressure value 312 that is data communicated over a data channel to a computing device 200, as further described below. The temperature sensor 113 may measure a temperature at one or more locations of the pressure head 10, either to determine a temperature of the patient 2 at the tissue, or to determine a temperature of the pressure head 10 that may be under temperature control. The temperature sensor 113 may comprise a thermocouple and/or infrared radiation sensors. The temperature sensor 113 may generate a temperature value 313 (abbreviated “temp. value 313″ in FIG. 9), which may for example be stored as degrees Fahrenheit or Celsius. Alternatively, or in addition, temperature value 318 may measure a temperature setting of the pressure head 10 which the practitioner may have selected for use (e.g., “high”, “medium,” or “low”, which may be independent of the actual temperature of the pressure head 10 and/or tissue of the patient 2).

The head type sensor 115 may detect a head type of the pressure head 10 placed on the lever arm 12. The head type sensor 115 may use an optical recognition unit to read a bar code, QR code, or other visual indicator. Alternatively, or in addition, other techniques know in the art may be utilized for sensing the head type, for example electromagnetic sensing (e.g., nearfield communication (NFC) and/or RFID tags). The head type sensor 115 may generate a head type value 317 that may be communicated to the computing device 200.

The timer 114 may be a timer that measures a time over which pressure is applied. The timer 114 may be linked to the pressure sensor 112, such that the timer activates when a threshold pressure is reached and/or released. For example, the timer 114 may activate when a pressure of at least five pounds per square inch is detected on the pressure head (e.g., 5 psi, and/or a force of 3 pounds), and deactivate when pressure less than five pounds per square inch is detected. The timer 114 may generate the time value 314. Although the timer 114 is illustrated as a treatment sensor 110, it should be noted that the timer 114 may be implemented on the computing device 200 or another data processing system. For example, a data processing system may record the time over which a continuous signal is received from the pressure sensor 112 over the threshold pressure and/or threshold force to determine the time over which the pressure treatment occurs, where a general clock of a computer processor or other timing circuit may provide the time value 314.

The vibration sensor 118 may detect a vibration level of the pressure head 10 to generate a vibration value 318. The vibration value may measure the amount of vibration in the pressure head 10 during a pressure treatment, and/or a vibration level which the practitioner may have selected for use. For example, five vibration modes may be available, each with different frequencies and/or sinusoidal profiles. The vibration value 318 may log such selection, regardless of the actual vibration experienced by the pressure head 10.

A head rotation sensor 116 may determine a rotation of the pressure head 10, for example during a pressure treatment, that may provide a rotational sheering to a tissue of the patient 2 (e.g., the rotational pressure 86). Depending on the type of pressure head 10, and whether it may exhibit a purposeful asymmetry in shape, the head rotation sensor 116 may also indicate which part of the pressure head 10 comes in contact with the tissue of the patient 2. The head rotation sensor 116 may generate the head rotation value 316 (abbreviated the “head rot. value 316 in FIG. 9). Head rotation may be sensed by optical sensors (e.g., detecting rotation through alternating contrast striping), magnetic sensors, and/or other techniques known in the art.

The treatment device 100 may include a set of one or more axis sensors 130. Each axis sensor of the axis sensors 130 may detect an aspect of axis adjustment that may affect the location of engagement and/or angle of engagement of the pressure head 10 on the body of the patient 2. A y axis rotation sensor 138 may measure the y axis rotation 38 of the third hinge point 37 to generate a y rotation value 337. A z axis rotation sensor 135 may measure the z axis rotation 36 of the second hinge point 35 to generate a z rotation value 335. An x axis translation sensor 133 may measure the x axis translation (e.g., along the sliding track 22) to generate an x location value 333. And a z-axis translation sensor 136 may measure the z axis translation (e.g., elevation and/or extension of the support 18, or other mode of height increase of the first hinge point 31 and/or hinge 16) to generate a z location value 336. Rotation and/or translation values may be sensed by optical sensors (e.g., detecting rotation through alternating contrast striping), magnetic sensors, and/or other techniques known in the art.

The treatment device 100 may also include one or more arm sensors 140 that may sense the position of the pressure head 10 along the lever arm 12 (and/or the head support arm 13), and/or sense the position of one or more grips 14. In one or more embodiments, the arm sensors 140 may include a head position sensor 142 that senses a location of the pressure head 10 along the lever arm 12 (and/or the head support arm 13) to generate the head position value 342. The head position value 342 may be sensed by optical sensors (e.g., detecting rotation through alternating contrast striping), magnetic sensors, and/or other techniques known in the art.

The treatment device 100 may be connected to a computing device 200, either through a network connection and/or a network 150. In one or more embodiments, the computing device 200 may display realitme data to aid the practitioner and/or the patient during the treatment session. In one or more embodiments, the computing device 200 may store data from the treatment session in a session data 300 for logging, analysis, and/or for addition to a medical chart of the patient 2. In one or more embodiments, the computing device 200 may recall session data 300 of previous sessions for the practitioner to assess prior to beginning a new treatment session, and/or to act as a “prescription” or guideline for the new treatment session.

The network connection may be a wired connection, a wireless connection, a Bluetooth® connection, or another connection. The network 150 may be a local area network (LAN), a local wireless internet network (WiFi network), a wide area network (WAN), and/or the Internet. The computing device 200 may comprise a microcontroller (e.g., a PIC microcontroller), a general purpose computer (e.g., a PC), a smartphone (e.g., an Android device, an iPhone®), a tablet device, a server computer (e.g., located in a data center), and/or another type of computing device. In one or more embodiments, the computing device 200 may be implemented with two or more computers and/or computing devices.

In one or more embodiments, the treatment device 100 generates a sensor data 201 which may be communicated to the computing device 200. The computing device 200 may then present all or a portion of the sensor data 201 on a speaker 170 and/or a display 160. The speaker 170 may be a speaker integrated into the computing device 200, or may also be connected through a network connection or the network 150 (e.g., a Bluetooth speaker, a WiFi connected speaker, etc.). For example, a recorded or synthetically generated voice may state certain parameters, metrics, and/or measurements of the sensor data 201, or warn of exceeding certain limits. For example, after a substantially static pressure is established on the pressure head 10 (e.g., fluctuating ±5% over 500 milliseconds), a voice may state the pressure (e.g., in psi, kPa) of the pressure on the pressure head 10 as communicated by the pressure sensor 112. Sounds, chimes, and other audible cues that can be learned and/or interpreted by the practitioner (but not necessarily by the patient, without training) can also be utilized. For example, a certain tone may specify a correct target pressure of the pressure head 10 against the tissue has been achieved, whereas a lower frequency tone may indicate that too little pressure is being applied and a higher frequency tone that too much pressure is being applied.

Similarly, the sensor data 201 or portions thereof may be displayed on a display 160. The display 160 may be a display screen for displaying number, letters, and/or graphical user interfaces. The display 160, for example may be a television, monitor, LED screen, LCD screen, digital display, or other electronic display. For example, the display 160 may be a smartphone screen where a mobile application displays aspects of the sensor data 201. In another example, the computing device 200 may be a PC and the display 160 may be an LCD screen, for example mounted on a wall behind the massage table where the practitioner can easily see the display and any sensor data 201 while providing a treatment session.

The realtime display routine 202 may receive sensor data 201 and may determine, according to a configuration file (not to be confused with the configuration data 337), which sensor data 201 to present and/or display in realtime. In one or more embodiments, the realtime display routine 202 comprises computer readable instructions that when executed receives the sensor data 201, optionally references a configuration file, and presents a selected subset data on the speaker 170 and/or the display 160 in a format matching the configuration file. For example, depending on the size of the display 160, or what is of importance for the patient 2 for a certain type of treatment, the realtime display routine 202 may emphasis pressure of the pressure head 10 and head rotation (e.g., as measured by the head rotation sensor 116). Certain pressure heads 10, when detected (e.g., by the head type sensor 115), may also change the data presented by the realtime display routine 202. The configuration profile may have custom parameters extracted from or otherwise reading a user profile of the patient 2 (e.g., a user profile stored in the patient database 208 of FIG. 9).

A session recording routine 204 may receive the sensor data 201 and store all or a subset of the session data 300 in computer memory (e.g., RAM, a hard disk, a solid state memory), for example within a session database 208 containing two or more instances of the session data 300 that may be linked to one or more instances of the user profile of the patient 2. Where the computing device 200 comprises a server (e.g., the computing device 200B, described below), the session database 208 may be stored at a location remote to the treatment device 100 and may be accessed over the network 150.

The session data 300 may comprise data detected during the treatment session, input by the patient 2, input by the practitioner, and/or data extracted from the sensor data 201. The session data 300 may be stored in a common data format, including a relational table and/or a JSON file. In one or more embodiments, the session data 300 comprises a patient UID 301 which may be a unique identifier of the patient 2 and/or a user profile representing and/or associated with the patient 2 (e.g., a globally unique identifier, a GUID). A practitioner UID 302 may similarly be a unique identifier of the practitioner and/or a user profile representing and associated with the practitioner. Although not shown, a data and time, a facility UID, and other metadata may be also stored in the session data 300.

The session data 300 may comprise one or more instances of the pressure treatment data 330. The pressure treatment data 330 may represent the data associated with one pressure treatment, for example one depression of the lever arm 12 (and/or the head support arm 13) to apply pressure to a tissue of the patient 2. Each pressure treatment data 330 may have data automatically upon a certain condition (e.g., detection of a certain threshold pressure) and end recording upon a certain different condition (e.g., release of pressure on the pressure head).

The pressure treatment data 330 may include a configuration data 337 that may include data attributes for storing values that specify a spatial arrangement of the treatment device 100 and/or the pressure head 10 in physical space (e.g., within the axes 30). For example, the configuration data 337 may include the y rotation value 338, the z rotation value 335, the x location value 333, and/or the z location value 336, as may be generated by the axis sensors 130 and as each are described above. The head data 315 may include attributes that store values for specifying a type and/or a relative position of the pressure head 10. For example, the head data 315 may include the head type value 317 and the head position value 342.

A pressure data 304 may include data attributes storing data values related to the time proximate to and/or during the pressure treatment. The pressure data 304 may include, for example, the pressure value 304, the time value 314 of the pressure and/or the pressure treatment (e.g., the pressure treatment 80), the vibration value 318, the temperature value 313, and/or the head rotation value 316, as each may be generated by the treatment sensors 110 and as each are described above. Multiple data values may be stored as a data set. For example, the pressure value 312 and the time value 314 may be stored as a pressure-over-time dataset, and/or a set of values describing a pressure curve that may begin data recording upon a certain condition (e.g., detection of a certain threshold pressure) and end recording upon a certain different condition.

The body data 306 may include data that may specify a body location and/or position of the patient 2 relative to the treatment device 100 and/or the surface 19 that may have a known and/or defined association with the treatment device 100. A location description 308 may be a description entered and/or audibly dictated by the practitioner. For example, the practitioner may state the position of the patient 2, which may be transcribed by a voice-to-text application, plugin, and/or remote procedure call. The location description can also, or alternatively, specify a location of the patient 2 on a surface with calibration marks. For example, a patient 2 may be directed by visual markers on the surface 19 (and with the assistance of the practitioner) to lie in a certain position on the surface 19, and that configuration of the patient 2 may be noted in the location description 308. Alternatively, or in addition, the location data 309 may be data obtained from a location calibration process (e.g., touching the pressure head 10 at different parts of the body of the patient 2 at the start of the treatment session and/or the beginning of each treatment subsession).

Although not shown in the embodiment of FIG. 1B, the computing device 200 may be one or more computing devices. For example, a client instance of the computing device 200 (e.g., a computing device 200A) may be a smartphone running a mobile application that comprises the realtime display routine 202, the session recording routine 204, and the session execution routine 206, and which receive the sensor data 201 (e.g., over a Bluetooth® connection to the treatment device 100) and generates an instance of the session data 300. Continuing the same example, a server instance of the computing device 200 (e.g., a computing device 200B) may be accessible over the network 150 and store the patient database 210 and the session database 208, as well as respond to storage requests to store the session data 300 and/or respond to queries to retrieve the session data 300. The computing device 200B may include additional server-side support for many instances of the treatment device 100 across multiple businesses, including for example user profiles of each patient 2 that may be securely segregated between businesses on dedicated and/or multitenant computing systems.

A session execution routine 206 may retrieve a session data 300 (e.g., from a current treatment session, from a previous treatment session) and load the session data 300 or portion thereof into a computer memory of the computing device 200 such that it can be presented to the practitioner and/or the patient 2. In one or more embodiments, the session execution routine 206 may query the session database 208 to retrieve a session data 300 selected by the practitioner from a user interface. In one or more embodiments, the session execution routine 206 may “playback” the session data 300 as a prescribed treatment, which the patient 2 and/or practitioner may have liked and/or found successful in their last treatment. This may allow the practitioner to substantially copy and/or repeat the previous treatment session, treatment subsessions, or portions thereof. In such example, the practitioner of the current treatment session may or may not be different than the one who originally administered the treatment session from which the session data 300 was generated. This may allow for higher consistency between treatment sessions, practitioners, facilities, and/or providers.

The session execution routine 206 may also retrieve prescribed sessions. The prescribed treatment session, as stored as data, may have a similar data structure to the session data 300. For example, the prescribed session data 300 may comprise one or more pressure treatment data 330 that the practitioner may be instructed to achieve, including various audible or visual cues to assist the practitioner to achieve the desired targets (including instructions to change instances of the pressure head 10, reposition the patient 2 on the surface 19, etc.). In one or more embodiments, the session execution routine 206 may (i) read a pressure treatment data 330 from a prescribed session data 300 and present it on a speaker 170 and/or a display 160 for the practitioner; (ii) determine one or more target values (e.g., target values of the treatment sensors 110, the axis sensors 130, and/or the arm sensors 140) have been achieved; (iii) generate a signal indicating the one or more target values have been achieved; and (iv) recording the pressure treatment data 330 of the actual pressure treatment that achieved the targets (e.g., via a procedure call to the session recording routine 204).

FIG. 10 is a data acquisition process flow 1000, according to one or more embodiments. Operation 1000 generates a session data object that may represent a treatment session of a patient 2. The session data object, for example the session data 300 of FIG. 9, may be initiated with a patient UID 301, two or more instances of a practitioner UID 302, a date, a time, a facility UID, a provider UID, and/or other data that may be automatically assigned and/or manually entered. Operation 1002 optionally initiates a subsession. A divider or other data grouping may be designated to group the data from a number of related pressure treatments (e.g., pressure treatments 80). For example, the subsession may group pressure treatments relating to a certain body part (e.g., muscle group), a certain body area (e.g., the back), a certain injury (e.g., an upper arm and shoulder injury), and/or other groupings that may be prudent within the expertise of the practitioner. As just one example, the pressure treatment 80.01 through the pressure treatment 80.12 of FIG. 11 and FIG. 12 may be defined as a treatment subsession.

Operation 1004 enters and/or confirms a patient position of the patient 2. For example, the practitioner may state a position of the patient 2 into a voice interface, or may press a button to confirm that the patient 2 has not moved from a previous position. Alternatively, or in addition, operation 1004 may be “opt-in”, wherein the position of the patient 2 will be assumed to be the same if the practitioner does not change the position data (e.g., the computing device 200 may read the previous position). Operation 1004 may store the body data 306, including the location description 308 and/or the location data 309.

Operation 1006 receives an entry specifying spatial parameters of, and/or detects axes parameters of, the treatment device 1 and/or the pressure head 10. For example, the practitioner may manually enter or read off parameters for the spatial configuration of the treatment device 1, and/or they may automatically detected as described in conjunction with the configuration data 337 of FIG. 9. Similarly, operation 1008 may receive an entry for, and/or detect, arm parameters of the lever arm 12 (and/or the head support arm 13) and/or the pressure head 10. For example, operation 1008 may receive and/or automatically detect a type of the pressure head 10, a location of one or more pressure heads 10 along the lever arm 12 (and/or the head support arm 13), and/or a location of one or more grips 14 along the lever arm 12.

Operation 1010 applies the pressure treatment (e.g., the pressure treatment 80). The pressure treatment may comprise an application of pressure by the pressure head 10 to a tissue of the patient 2. The pressure treatment may be applied by the practitioner to the patient 2 as a result of the practitioner applying force to the effort portion of the lever arm 12. Alternatively, or in addition, the pressure treatment may be applied by a power source (e.g., the actuator 74). Operation 1012 measures one or more treatment values. The treatment values, for example, may include the pressure data 304, the pressure value 312, the time value 314, the vibration value 318, the temperature value 313, the head rotation value 316, etc. The treatment values may be optionally displayed to the practitioner or otherwise presented (e.g., audibly). Operation 1014 may then record a treatment data point in a computer memory. For example, the treatment data point may be the pressure treatment data 330 within the session data object (e.g., the session data 300).

Operation 1016 determines whether another pressure treatment is to occur within the subsession (or, in the event in which no subsession has been designated, within the treatment session). If another pressure treatment is to occur within the subsession, operation 1016 may return to operation 1004, where the patient 2 may be repositioned and/or confirmed to be in the same position. If no further pressure treatment is to be effected, operation 1016 may proceed to operation 1018. The determination of operation 1016 may occur automatically (e.g., detection that the practitioner is preparing for another pressure treatment through the sensor data 201) and/or manually (the practitioner entering data specifying, or otherwise indicting, that another pressure treatment is to occur).

Operation 1018 determined whether another subsession is to be initiated. If an additional sub-session is to be initiated, operation 1018 returns to operation 1002. Otherwise, operation 1018 may end and the treatment session may terminate. A determination that another subsession is not to occur in operation 1018 may also initiate the solidification, storage, and/or upload of the session data object, for example to a remote server for later retrieval.

An example treatment session utilizing the treatment device 1 will now be described in the embodiments of FIG. 11 through FIG. 14. In the embodiments of FIG. 11 through FIG. 14, each closed shape (generally squares, rectangles, circles, and ellipses), is used to represent the contact point and/or “footprint” of a pressure treatment 80. The particular shape of the contact point may be a result of different pressure head 10 having matching contact surface. (However, where noted, certain elliptical closed shapes may also designate a circular point rotated to demonstrate pressure application to a tissue perpendicular to the surface 19). Each arrow drawn to the pressure treatment 80 may represent a pressure vector 82, and may include a linear pressure 84 or a rotational pressure 86. A pressure vector 82 having a triangular unshaded tail may designate that that pressure vector 82 is substantially normal (e.g., ±5 degrees, ±10 degrees) to the tissue of the patient 2 at a central point of contact of the pressure head 10 (which may otherwise have been drawn as coming “out of” the page of the figure). A dashed line may represent light pressure, for example for use in mainly applying heat, cool, vibration, and/or light rotation at the surface of the skin. Sheering pressure may be established by a linear pressure that is at an angle other than normal to the tissue of the patient at the point of the pressure treatment 80 (e.g., greater than ±5 degrees from normal). Rotational sheering pressure may be established by the rotational pressure 86 which may be simultaneously administered with a linear pressure 84. The amount of pressure applied in each pressure treatment, and additional parameters (e.g., vibration, heat) are described but could be further visualized by texturing or shading the closed shapes.

FIG. 11 is an example treatment session 1100, according to one or more embodiments. FIG. 11 demonstrates use of the treatment device 1 to treat a shoulder injury of a patient 2. For example, the shoulder injury may be a rotary cuff injury. The injury may have resulted, for example, from a sports injury (e.g., tennis), an occupational injury (e.g., lifting a heavy object), an accident (e.g., tripping and falling), or a recreational injury (e.g., water skiing). The practitioner may complete a patient intake process and may be handed a diagnosis by a different medical provider and/or may directly diagnose the patient 2. Following the diagnosis, or a general understanding of pain, inflammation, tightness, or other conditions in which manual pressure may be beneficial, the practitioner may decide on a general treatment strategy that is adaptive during the treatment session, and/or may select a prescribed treatment. The prescribed treatment may be based on previous treatment records of the patient 2 and/or other patients 2 with similar injures, as may be available by query to the session database 208).

FIG. 11 illustrates a first portion of an example treatment session 1100 in which the patient 2 is placed on a surface 19 in a face-up position. The patient 2 may have removed their shirt or may have their remained clothed, depending on their treatment needs, the preference of the patient 2, the preference of the practitioner, the type of pressure heads 10 utilized, and/or other factors. As described in conjunction with the embodiment of FIG. 9, the practitioner may initiate a treatment session and optionally designate a subsession for the shoulder of the patient 2. The practitioner may then spatially configure the treatment device 1, the lever arm 12, the head support arm 13, and/or the pressure head 10 to carry out the pressure treatment 80.1, which may be at a slightly off-normal angle relative to the upper shoulder. The practitioner, for example, may apply 125 psi. The patient 2 may or may not participate in the treatment parameters of the pressure treatment 80.01. For example, the patient 2 may give feedback regarding pain, discomfort, and/or relief that may influence the pressure treatment 80.01 or other pressure treatments 80.

The practitioner may replace the pressure head 10 with a different pressure head 10 having a rectangular contact surface and reconfigure the treatment device 1 and/or reposition the patient 2. For example, to reposition the treatment device 1 such that the pressure head 10 contacts the tissue at the location of the pressure treatment 80.02, rather than remain at the location of the pressure treatment 80.01, the practitioner may effect an x axis translation along the x axis 30x that may run along an edge of the surface 19, may “scoot” the patient inward on the surface 19, and/or adjust the location of the pressure head 10 along the lever arm 12 (and/or the head support arm 13). The pressure treatment 80.02 may apply pressure to a pectoral region and/or muscle group of the patient 2. The practitioner may apply three separate instances of pressure at three different angles: (i) a first instance of the linear pressure (e.g., the linear pressure 84.01A, not labeled) that is normal to the tissue of the patient 2 and also includes rotational pressure 86; (ii) a second instance of the linear pressure (e.g., the linear pressure 84.01B, not labeled) that is at 45 degrees to the tissue of the patient 2 (e.g., from a 45 degree or clockwise instance of the y axis rotation 38), and a (iii) third instance of the linear pressure (e.g., the linear pressure 84.01C, not labeled) that is at -45 degrees to the tissue of the patient 2 (e.g., from a -45 degree or counterclockwise instance of the y axis rotation 38). The pressure head 10 utilized for the pressure treatment 80.02 may have a different surface for increased friction with the skin and/or clothing of the patient 2, for example a surface comprising silicone rubber or urethane. Alternatively, a pressure head 10 with a lower coefficient of friction pressure heads 10 may be utilized for certain pressure treatments 80, for example stainless steel, plastic, or Teflon. The practitioner may then replace the pressure head 10 again to carry out the pressure treatment 80.03 (e.g., a shorter rectangular pressure head 10), and yet again to carry out the pressure treatment 80.04 to tissue above the pectoral minor muscles, the pressure treatment 80.05 to tissue above the deltoid muscles, and the pressure treatment 80.06 to tissue above the bicep muscles (e.g., each completed with a pressure head 10 having a circular contact surface). The pressure head utilized for the pressure treatment 80.04, the pressure treatment 80.05, and the pressure treatment 80.06 may have a different shore hardness (e.g., softer or harder) than the pressure heads 10 utilized for the pressure treatment 80.1, the pressure treatment 80.2, and the pressure treatment 80.3.

In one or more embodiments, multiple pressure vectors 82 at the same tissue location with the same pressure head 10 may be treated as a single pressure treatment 80 and/or discrete pressure treatments 80. For example, it should be noted that while three linear pressures 84 are applied to the pressure treatment 80.02, this may comprise either a single pressure treatment 80 as shown in FIG. 11 or three distinct pressure treatments 80. Depending on how configured, any data recorded may be stored in the same pressure treatment data 330 or three distinct instances of the pressure treatment data 330. For simplicity of explanation in the embodiments of FIG. 11 through FIG. 14, multiple instances of a pressure vector 82 at the same location with the same pressure head 10 are treated as a single instance of the pressure treatment. In such case, the pressure treatment data 330 may store pressure data 304 for all of the individual depressions of the pressure head 10 (e.g., storing a pressure value 312A, a pressure value 312B, a pressure value 312C, etc.), or may average the values.

FIG. 12 illustrates a continuation of the example treatment session 1100 of FIG. 11, according to one or more embodiments. The practitioner may reposition the patient 2 to be face down against the surface (e.g., back up toward the pressure head 10 in its default spatial configuration), and apply six additional instances of the pressure treatment 80, including the pressure treatment 80.07 to tissue above the trapazoid muscles, the pressure treatment 80.08 to tissue above the pectoral major muscles, the pressure treatment 80.09 to tissue above the pectoral major muscles, the pressure treatment 80.10 to tissue above the pectoral minor muscles, the pressure treatment 80.11 to tissue above the bicep muscles, and the pressure treatment 80.12 to tissue above the road facia sheer muscles. The pressure head 10 utilized for the pressure treatment 80.07 may be circular, but is shown at an angle parallel to the top of the shoulder (which may be perpendicular to the surface 19 against which the patient 2 may be positioned). Following the treatment subsession, the patient 2 may be inspected, tested, evaluated, and/or asked whether pain or discomfort persists, in which case certain pressure treatments 80 may be repeated. Where the patient 2 may have an unrelated knee injury, the practitioner may initiate a new subsession.

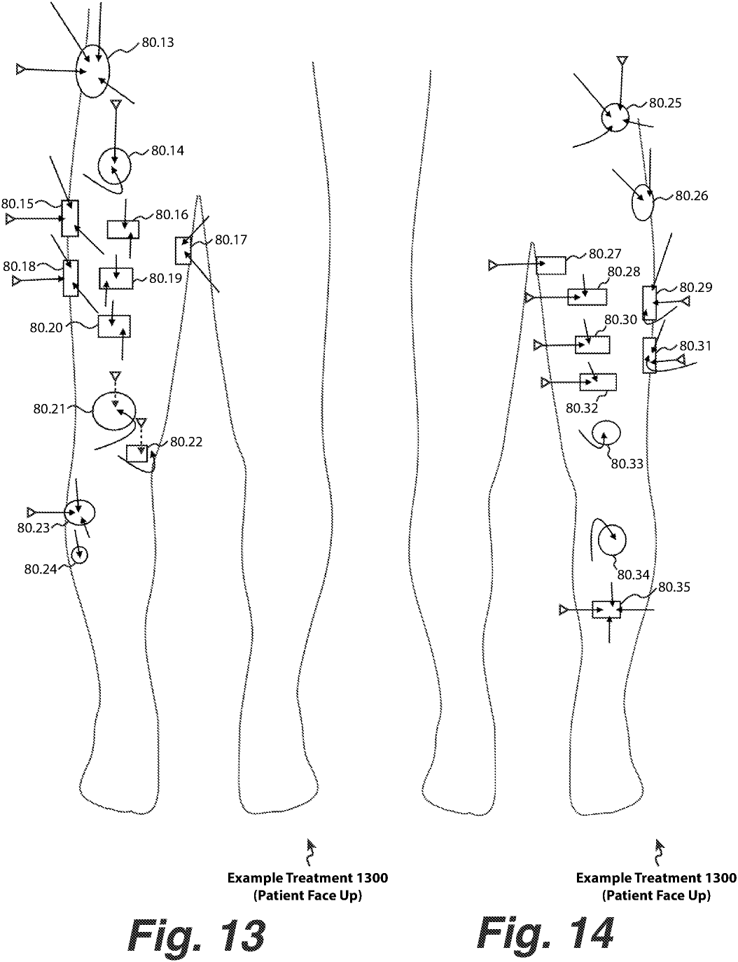

FIG. 13 illustrates a treatment session 1300 that may be a treatment session and/or a treatment subsession for a knee injury, according to one or more embodiments. The patient 2 may be first positioned against the surface 19 in a face up configuration. The embodiment of FIG. 13 may follow a similar routine to the embodiment of FIG. 11, in which various tissue locations are identified and treated by the practitioner, possibly with multiple instances of the pressure head 10. Directly above the knee, pressure treatment 80.21 may apply light pressure (e.g., 150 psi to 200 psi), while providing the rotational pressure 86. Other noteworthy pressure treatments 80 include the pressure treatment 80.13 in which the TFL is treated with 200 psi to 300 psi. FIG. 14 illustrates a continuation of the treatment session 1300 of FIG. 13, in which the patient 2 is placed face-down relative to the surface 19. Other noteworthy pressure treatments 80 include the pressure treatment 80.28 in which hamstring tendons may be treated.

In one or more embodiments, a treatment method may include testing an elasticity, degree of physical depression, and/or shore hardness of the tissue of the patient 2. For example, the pressure head 10 may be placed on the tissue of the patient 2 and a measurement of the tissue’s ability to be compressed may be taken. For example, 50 psi may be applied and it may be determined that the tissue compresses 15 millimeters. Following treatment, the same location may be re-tested to determine if the tissue has changed (e.g., loosened, decreased in shore hardness), for example yielding 22 millimeters. Conversely, the pressure required to depress the tissue a certain distance may be measured. Data can be collected and stored, for example in the session database 208. The data may be stored in association with and/or separately with respect to the pressure treatment data 330, for example as a tissue test data within the session data 300. In general, loosening tissue may indicate progress in healing. Objective and consistent methods of treatment of the tissue, using one or more of the present embodiments, may further assisting in evaluating effectiveness studies, communicating replicable treatments between persons and organizations, effective training by following objective instructions (e.g., rather than purely through experiential hands-on tissue manipulation), and/or the establishment of standard industry guidelines..

FIG. 15 illustrates an example embodiment of the pressure treatment device 1 in which a head support arm 13 may be actuated to apply the pressure to the tissue of the patient 2 by either a force generator 11 such as an off-axis instance of a cam 92 operated by a motor 90 that may be controlled by the practitioner and/or a lever arm 12 on which the practitioner may apply manual pressure, according to one or more embodiments. For clarity, the support 18 is shown in transparency with an outline in dotted lines. The support comprises an opening 59 creating a channel in which the head support arm 13 and the force support 98A may slide, as further described below. The opening 59 is shown in transparency in dashed lines along a surface of the support 18.

In one or more embodiments and the embodiment of FIG. 15, two functional units may be defined and coupled to one another, for example through the recoil element 23: (i) a force generating unit and (ii) a pressure application unit. In the present embodiment, the force generating unit may comprise a carriage 96, a force support 98A, a force support 98B, a lever arm 12, a cam 92, and a motor 90. The force generating unit may apply force to the head support arm 13.

The carriage 96 may be a sled or carriage system allowing for translation along and/or within the support 18, for example where the support 18 is implemented with square metal stock as may be the case in FIG. 4 through FIG. 6. The carriage 96 may run along one or more bearings 97, presently the bearing 97A and the bearing 97B. The carriage 96 may be coupled to the force support 98A and the force support 98B. The force support 98A may be coupled to the lever arm 12, for example using a hinge point and/or pin (e.g., which may be similar to the first hinge point 31 of FIG. 4). The lever arm 12 may include the contact 94A that may protrude to apply force to the contact 94B when the lever arm 12 is engaged. The force support 98A and the force support 98B may be separate components that are joined or coupled, and/or a single machined component. The force support 98B may include a through-hole (not shown) which may receive a drive shaft (not shown) from the motor 90. The motor 90 may be an electric motor, for example a stepper motor or servo motor. The motor may be powered by a power supply, for example a battery or wired outlet (not shown). The motor may include a gear reduction or gearbox to control speed and/or power of the cam 90. The motor 90 may be controlled by a control of the practitioner, for example a handheld remote, foot pedal, a mobile phone software application (e.g., an “app”), and/or a button on a grip 14 of the lever arm 12. The drive shaft may extend through the through-hold of the force support 98B into the cam 92.

The cam 92 may be a circle, ovoid, or other suitable shape that when rotated applies force from a surface of the cam 92 to the contact 94B. For example, the cam 92 may be a circle where the drive shaft is placed off-center. In one or more embodiments, and the embodiment of FIG. 15, the cam 92 may be an ovoid with an off-center receiver of the drive shaft. The force support 98A may define a partial circle (e.g., a quarter-circle) that may be matched with the cam 92 as to provide a stop and/or a brake in the rotation of the cam 92. In one or more other embodiments, the cam 92 may be allowed to freely rotate, which may cause a sinusoidal or other periodic fluctuation in the z axis value, in turn usable to apply a predetermined treatment length and/or a repeated treatment. This may be useful, for example, for applying a repeated pressure treatment in which pressure is applied every 10 seconds for fifty iterations. In one or more embodiments, a stop or break may be configurable to control the distance in which the pressure head 10 may travel. In one or more embodiments, the cam 92 may be replaceable such that different profiles of pressure treatment, including vibration, may be generated. For example, other instances of the cam 92 may be an irregular shape or undulating surface. The contact 94B may be wide enough to accommodate both the application of force from the he cam 92 and/or the contact 94A of the lever arm 12.

The pressure application unit may include the carriage 100, the head support arm 13, the contract 94B, the head bracket 46, the head extender 44, and the pressure head 40. The pressure application unit may receive the force from the force generating unit may convey force to the pressure head 40 to apply the pressure treatment to the tissue of the patient 2. The carriage 100 may similarly include one or more bearings 101, specifically in the embodiment of FIG. 15 a bearing 101A and a bearing 101B. As previously described, the head support arm 13 may include a track 15B permitting translation of the head bracket 46 (e.g., following depression of the adjustment button 48).

The force generating unit may be mechanically coupled to the pressure application unit, for example with a spring 99. The spring 99 may be selected such that the pressure application unit is stable (e.g., not bouncing) but moves readily and does not absorb substantial energy when the force from the force application unit is applied. Constraining the carriage 97 and/or the carriage 100 within the support 18 may assist translating the force generated by the force generation unit into a linear motion of the head support arm 13, the translation occurring down and along the opening 59 (e.g., along the z axis 30).

Both the force generation unit and the pressure application unit may be suspended along and/or within a hollow instance of the support 18 by a z-axis adjustor. In the embodiment of FIG. 15, the force generation unit and the pressure application unit are shown inside a hollow of the support 18. The z-axis adjustor may be a mechanism that allows for movement down the length of the support 18. In one or more embodiments and the embodiment of FIG. 15, both the force generation unit and the pressure application unit may be suspended by the extension rod 79 of the actuator 74. The actuator 74 may be therefore utilized to adjust a height of the pressure head 40 above the patient 2. It can be noted that in one or more embodiments the extension rod 79 of the actuator 74 could be directly connected to the pressure application unit and used to generate the force on the pressure head 40. However, in one or more embodiments it may be advantageous to have a slower, more powerful drive for the actuator 74, including to support the weight of the mechanics. In contrast, in one or more embodiments, the force generation unit may allow for a faster application of pressure that may be desirable by the practitioner, and may also enable a tactile response that may help assess the pressure treatment as it is applied.

As a result of one or more of the present embodiments, the practitioner may be able to apply high pressure to a target tissue and/or retain strength through multiple sessions without resting. This may allow each patient 2 to receive more quality care, and therefore heal faster and more effectively. It may also allow the practitioner to treat more people each day, and earn more money for themselves and/or an associated medical practice. As a result of one or more of the present embodiments, the practitioner (and/or a different practitioner) may be able to apply consistent pressure from one person to another, one treatment session to another, and/or even across organizations (e.g., where session database 208 is accessible to the patient 2 and/or accessible across the organizations). As a result of one or more embodiments, objective studies on the effectiveness of pressure treatments may be able to be performed, especially where data can be easily recorded and pressure treatments easily reproduced. This may increase the rate of healing, increase the number of people healed, reduce costs by avoiding surgery, increase the satisfaction of the patient, and the flexibility of the workforce of a medical provider which may increase profitability. As a result of one or more of the present embodiments, for example and without limitation the embodiment of FIG. 9, it may be relatively easy for the practitioner to quantify, record and/or communicate manual pressure treatments, including to improve quality across multiple treatments, practitioners, and/or organizations. Similarly, as a result of one or more of the present embodiments, the patient 2 and/or the practitioner may be able to focus on the treatment itself while receiving feedback and collecting data to ensure pressure treatments are consistent and correct (e.g., designated angles in the third hinge point 37, realtime data feedback on the display 160, queried data from past treatment sessions stored in the session database 208 that can be used prescriptively, etc.).

Although the present embodiments have been described with reference to specific example embodiments, it will be evident that various modifications and changes may be made to these embodiments without departing from the broader spirit and scope of the various embodiments. For example, the various devices, engines, agent, routines, and modules described herein may be enabled and operated using hardware circuitry (e.g., CMOS based logic circuitry), firmware, software, or any combination of hardware, firmware, and software (e.g., embodied in a non-transitory machine-readable medium). For example, the various electrical structure and methods may be embodied using transistors, logic gates, and electrical circuits (e.g., application specific integrated circuitry (ASIC) and/or Digital Signal Processor (DSP) circuitry).

In addition, it will be appreciated that the various operations, processes, and methods disclosed herein may be embodied in a non-transitory machine-readable medium and/or a machine-accessible medium compatible with a data processing system (e.g., the computing device 200). Accordingly, the specification and drawings are to be regarded in an illustrative rather than a restrictive sense.

The structures in the figures such as the engines, routines, and modules may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the preceding disclosure.

Embodiments of the invention are discussed above with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures.

From reading the present disclosure, other variations and modifications will be apparent to persons skilled in the art. Such variations and modifications may involve equivalent and other features which are already known in the art, and which may be used instead of or in addition to features already described herein.

Although claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any claim and whether or not it mitigates any or all of the same technical problems.

Features which are described in the context of separate embodiments may also be provided in combination in a single embodiment. Conversely, various features which are, for brevity, described in the context of a single embodiment, may also be provided separately or in any suitable sub-combination. The applicants hereby give notice that new claims may be formulated to such features and/or combinations of such features during the prosecution of the present application or of any further application derived therefrom.

References to “one embodiment,” “an embodiment,” “example embodiment,” “various embodiments,” “one or more embodiments,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least one or more embodiments of the invention” includes the stated particular feature, structure, or characteristic.

The enumerated listing of items does not imply that any or all of the items are mutually exclusive, unless expressly specified otherwise.

It is understood that the use of a specific component, device and/or parameter names are for example only and not meant to imply any limitations on the invention. The invention may thus be implemented with different nomenclature and/or terminology utilized to describe the mechanisms, units, structures, components, devices, parameters and/or elements herein, without limitation. Each term utilized herein is to be given its broadest interpretation given the context in which that term is utilized.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; a smartphone, application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, one or more embodiments of the disclosure may be practiced in network computing environments with many types of computer system configurations, including personal computers, hand-held devices, multiprocessor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java.TM., Jini.TM., C, C++, Smalltalk, Perl, UNIX Shell, Visual Basic or Visual Basic Script, Virtual Reality Markup Language (VRML), ColdFusion. TM. or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system’s registers and/or memories into other data similarly represented as physical quantities within the computing system’s memories, registers or other such information storage, transmission or display devices.

The term “processor” may refer to any device or portion of a device that processes electronic data from registers and/or memory to transform that electronic data into other electronic data that may be stored in registers and/or memory. A “computing platform” may comprise one or more processors.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing the certification network 100 according to the present invention will be apparent to those skilled in the art. Various aspects of the invention have been described above by way of illustration, and the specific embodiments disclosed are not intended to limit the invention to the particular forms disclosed. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A device for massage, physical therapy, chiropractic therapy, and myofascial release, the device comprising:
a pressure head for applying a pressure to a tissue of a patient,
a lever arm comprising a first end and a second end coupled to the pressure head,
wherein the pressure head protruding below the lever arm and a force acting on the second end of the lever arm multiplies the force applied to the tissue by the pressure head,
a first hinge attached to the first end of the lever arm and configured to allow the pressure head to pivot into and out of contact with the tissue of the patient,
a support coupled to the first hinge elevating the first hinge above a fastening point, and
a fastener coupled to the support, for fixing the device when the pressure is applied to the tissue of the patient.

2. The device of claim 1, further comprising:
a track configured to translate of the device along a first axis, referred to as an x-axis, running parallel to a surface that is elongated and usable to receive the patient in a substantially flat position,
wherein the fastener fixes the device at a location in the track along the first axis.

3. The device of claim 2, further comprising:
a second hinge coupled to the support and configured to rotate the lever arm around a second axis, referred to as a y-axis, that runs perpendicular to the elongated surface for receiving the patient, the second hinge enabling rotation of the lever arm such that an oblique pressure can be applied from the pressure head to the tissue when the patient is positioned substantially flat on the surface, and
a third hinge coupled to the support and configured to rotate the lever arm around a third axis, referred to as a z-axis, that runs perpendicular to both the y-axis and the z-axis, the third hinge enabling rotation of the lever arm to apply the pressure to the tissue at two or more horizontal locations of the patient without the patient repositioning on the surface.

4. The device of claim 1, further comprising:
a first grip located on the lever arm at least one of at the second end of the lever arm and a first location between the pressure head and the second end of the lever arm, the first grip for receiving an extremity of a practitioner to apply the force acting on the second end of the lever arm,
wherein the first grip positioned such that the force applied to the tissue by the pressure head is an integer multiple of the force acting on the second end of the lever arm.

5. The device of claim 1,
wherein the fastener coupled to a surface that is a table on which the patient rests in the substantially flat position,
wherein the table comprising a head space comprising a cavity and a hole for receiving a head of the patient when the patient is positioned on their stomach on the surface, and
wherein a track extends along one edge of the table such that the pressure head may be translated to different locations along the table.

6. The device of claim 1, further comprising:
an attachment point located on the lever arm coupling the pressure head to the lever arm,
wherein the pressure head detachable from the lever arm such that at least one of a different pressure head and a replacement pressure head attachable to the attachment point.

7. The device of claim 1,
wherein the pressure head comprising a friction surface for engagement with skin over the tissue for gripping the skin during application of the pressure to provide a sheer pressure to the tissue, and
wherein the friction surface comprising at least one of a rubber, a silicone, and a polymer.

8. The device of claim 3, further comprising:
a second grip located on the lever arm between the pressure head and the first grip, the second grip for receiving an extremity of a practitioner to apply the force acting on the second end of the lever arm at a different force multiplier than the first grip,
a sensor measuring at least one of the pressure applied to the tissue of the patient and the force applied to the pressure head,
wherein the sensor transmitting data to at least one of a computer memory for storage and a user interface for display to the practitioner.

9. The device of claim 3,
wherein a length of the lever arm is adjustable to at least one of adjust a force multiplier and to permit a practitioner to remain in physical contact with the patient during treatment, and
wherein the lever arm comprising a second track allowing the pressure head to slide along the lever arm to at least one of adjust the force multiplier on the lever arm and reposition the pressure head enabling application of pressure to the tissue at different horizontal locations of the patient without the patient repositioning on the surface.

10. A method for bodily tissue manipulation, the method comprising:
positioning a patient on a surface;
placing a pressure head on a tissue of the patient
wherein the pressure head coupled to a lever arm pivoting on a fulcrum that is fixable relative to the surface, and
wherein the lever arm comprising an effort portion of the lever arm, a load portion of the lever arm, and the fulcrum comprises a pivot point;
applying a force to the lever arm at the effort portion of the lever arm; and
applying a multiplier force to the pressure head at the load portion of the lever arm to provide a pressure treatment for healing the patient that is able to be duplicated while reducing exertion on a practitioner applying the force to the lever arm.

11. The method of claim 10, further comprising:
translating the lever arm and the pressure head parallel to a vertical axis of the patient; and
immovably fixing the pressure head relative to the surface.

12. The method of claim 11, further comprising:
rotating the pivot point of the fulcrum around an axis perpendicular to a frontal axis of the patient,
wherein the force resulting in an oblique pressure applied by the pressure head.

13. The method of claim 12, further comprising:
rotating the pivot point of the fulcrum around an axis perpendicular to a sagittal axis of the patient to reposition a contact point of the pressure head to the tissue of the patient.

14. The method of claim 13, further comprising:
changing a force multiplier by adjusting at least one of a length of the effort portion of the lever arm, a position of the pressure head along the lever arm, and a position of the grip along the lever arm.

15. The method of claim 14, further comprising,
replacing the pressure head with a different pressure head; and
applying a different pressure treatment to the patient.

16-20. (canceled)

21. A device for applying a pressure treatment to a tissue of a patient, the device comprising:
a pressure head for applying a pressure to the tissue of the patient,
a head support arm coupled to the pressure head,
wherein the pressure head protruding below the head support arm and a force acting on the head support arm applies the force to the tissue through the pressure head,
a support coupled to the head support arm distancing the head support arm from the patient,
a z-axis adjustor coupled to the support and the head support arm, the z-axis adjustor configured to move the head support arm such that a distance of the pressure head to the tissue of the patient may be adjusted,
a force generator coupled to at least one of the support and the head support arm and configured to apply the force to the head support arm, and
a fastener coupled to the support, for fixing the device when the pressure is applied to the tissue of the patient.

22. The device of claim 21, wherein the force generator comprises a cam that when rotating applies the force to the head support arm.

23. The device of claim 22, wherein the force generator further comprises a lever arm coupled to the support, and wherein the z-axis adjustor comprises an actuator.

24. The device of claim 23, further comprising:
a first carriage moving on a first set of one or more bearings at least one of along the support and inside the support and coupling the support and the force generator,
a second carriage moving on a second set of one or more bearings at least one of along and inside the support and coupling the support and the head support arm, and
a recoil element elastically coupling the first carriage and the second carriage such that the force applied by the force generator causes the recoil element to elongate when the pressure is applied to the tissue of the patient and to recoil after the force ceases to be applied by the force generator.

25. The device of claim 24, further comprising:
a track configured to translate of the device along a first axis, referred to as an x-axis, running parallel to a surface that is elongated and usable to receive the patient in a substantially flat position,
wherein the fastener fixes the device at a location in the track along the first axis,
a second hinge coupled to the support and configured to rotate the lever arm around a second axis, referred to as a y-axis, that runs perpendicular to the elongated surface for receiving the patient, the second hinge enabling rotation of the lever arm such that an oblique pressure can be applied from the pressure head to the tissue when the patient is positioned substantially flat on the surface, and
a third hinge coupled to the support and configured to rotate the lever arm around a third axis, referred to as a z-axis, that runs perpendicular to both the y-axis and the z-axis, the third hinge enabling rotation of the lever arm to apply the pressure to the tissue at two or more horizontal locations of the patient without the patient repositioning on the surface.
